(19) United States
(12) Patent Application Publication
(10) Pub. No.: US 2023/0063876 A1
(43) Pub. Date: Mar. 2, 2023

(54) Virus-Like Particle Vaccines for Opioid Drugs

(71) Applicants: UNM RAINFOREST INNOVATIONS, Albuquerque, NM (US); ARIZONA BOARD OF REGENTS, FOR AND ON BEHALF OF NORTHERN ARIZONA UNIVERSITY, Flagstaff, AZ (US)

(72) Inventors: Bryce Chackerian, Albuquerque, NM (US); Kathryn Marie Frietze, Albuquerque, NM (US); Naomi Lee, Flagstaff, AZ (US); Susan Core, Los Ranchos, NM (US)

(21) Appl. No.: 17/796,368

(22) PCT Filed: Feb. 28, 2021
(86) PCT No.: PCT/US2021/020162

§ 371 Date: Jul. 29, 2022

Related U.S. Application Data
(60) Provisional application No. 62/984,123, filed on Mar. 2, 2020.

(51) Int. Cl. A61K 39/00; A61P 25/36

(52) U.S. Cl. CPC A61K 39/0013; A61P 25/36; A61K 2039/5258; A61K 2039/6075; A61K 2039/627

(57) ABSTRACT

The present invention is directed to virus-like particles (VLPs) preferably derived from Qbeta bacteriophage which are engineered to conjugate to derivatives of opioid drugs. The opioid drugs are conjugated at high density to the virus-like particles to achieve long-lasting and high titer antibodies to the drugs of interest. Methods of treatment are also described.

### RELATED APPLICATIONS

This application claims the benefit of priority of U.S. provisional application Ser. No. 62/984,123 of identical title filed Mar. 2, 2020, the entire contents of which application is incorporated by reference herein.

### FIELD OF THE INVENTION

The present invention is directed to virus-like particles (VLPs) preferably derived from Qbeta bacteriophage which are engineered to allow conjugation to derivatives of opioid drugs and to treat the effects of opioid addiction and related conditions and disease states related to opioid addiction. In this invention, the opioid drugs are conjugated at high density to the virus-like particles. These conjugated virus-like particles are assessed for immunogenicity (e.g. in mice) over a range of doses and immunization schedules to assess [1] the titers of antibodies elicited by the vaccines, [2] the longevity of the antibody response, [3] the optimal dosing and immunization schedule to achieve long-lasting and high titer antibodies to the drugs of interest.

### BACKGROUND AND OVERVIEW OF THE INVENTION

As the opioid crisis grows, novel treatments for opioid use disorder are desperately needed. Opioid vaccines that elicit high titer, long-lasting antibodies that block opioid drug activity are a promising treatment approach. However, eliciting high titer antibodies to a small molecule is challenging. Current vaccine strategies have focused on generating derivatives of opioid drugs such that they can be conjugated to an immunogenic protein carrier, such as KLH or tetanus toxoid (1). This classical conjugate approach for inducing antibodies to a hapten has shown moderate success in eliciting antibodies to hydrocodone, oxycodone, morphine, and fentanyl (2-6). However, conjugate vaccines are not highly immunogenic; they require repeated boosts and addition of adjuvants to elicit suitable titers, and they generally do not elicit long-lasting antibody responses.

The inventors have a long history of using bacteriophage virus-like particles (VLPs) to display poorly immunogenic molecules in high density arrays to the immune system such that high titer, long-lasting antibodies can be elicited with small doses and without repeated boosts (7-15). There is growing interest in using vaccines to treat opioid use disorder (OUD).^{1,2} An effective opioid vaccine would function by inducing anti-opioid serum antibodies that bind opioid drugs in the blood and prevent them from crossing the blood-brain barrier to exert their effects.^{1,2} Several candidate vaccines have shown promise in blocking opioid-induced anti-nociception, respiratory depression, and self-administration of drug in mice, rats, and non-human primate models^{1,2}

Opioids are not inherently immunogenic, meaning that they need to be attached to a more immunogenic scaffold in order to effectively elicit antibody responses. Previous efforts to develop vaccines have used classical strategies in which opioids are attached to protein carriers, such as keyhole-limpet hemocyanin (KLH) or tetanus toxoid (TT). Although these vaccines can elicit antibodies that bind opioids and block their activity in vivo, they typically require multiple immunizations to elicit sufficient antibody levels, and antibodies wane quickly without repeated boosts. To maximize its clinical utility, an ideal opioid vaccine would induce high antibody levels rapidly, ideally after a single vaccination, and these antibody responses would be long lasting. In the present invention, the inventors provide derivatives of opioid drugs such that they can be chemically conjugated at high density on VLPs derived from Qbeta and other bacteriophages as described herein.

The present invention is generally directed to opioid vaccines which display opioid drugs on a highly immunogenic Qβ bacteriophage virus-like particle (VLP)-based vaccine platform and methods of using these vaccines in the treatment of subjects to reduce the effects of opioid use disorder, opioid overdose, or to lessen the effects of, diminish or inhibit one or more symptoms of opioid use. The inventors provide VLP-based opioid vaccines which can elicit high-titer serum antibodies in as few as 7 days after a single intramuscular immunization, and show that mice are protected from a lethal overdose as soon as 20 days post immunization. Further, the antibodies which are produced by administering the immunogenic compositions pursuant to the present invention are long-lasting, often persisting for at least 120 days after a single immunization. The present invention provides develop vaccines capable of neutralizing the effects of commonly abused opioids.

### BRIEF DESCRIPTION OF AND RATIONALE FOR THE INVENTION

Bacteriophage virus-like particles (VLPs) are highly immunogenic vaccine platforms that are multivalent platforms that can be used to dramatically increase the immunogenicity of molecules that are normally poorly immunogenic. The present invention is directed to virus-like particles (VLPs) derived from bacteriophages, especially Qbeta or AP205 bacteriophage which are engineered to provide VLPs to allow conjugation to derivatives of opioid drugs. The opioid drugs are conjugated at high density to the virus-like particles. The opioid drugs are conjugated principally to lysine residues which exist on the surface of the VLP.

The present invention provides immunotherapeutic and prophylactic Qbeta bacteriophage viral-like particles (VLPs) conjugated to opioid derivatives which are useful in the treatment and prevention of opioid dependency, opioid use disorder and opioid overdose in subjects in need. Related compositions (e.g. immunogenic compositions, including vaccines) and therapeutic methods are also provided. VLPs and related compositions of the invention induce high titer antibody responses to protect against opioid dependency, opioid use disorder, opioid overdose and associated symptoms in subjects in need. VLPs. VLP-containing compositions, and therapeutic methods of the invention induce an immunogenic response against opioids, confer immunity against, protect against and reduce the likelihood of opioid use disorder, dependency, overdose and related symptoms as disclosed herein caused by opioid use, especially include opioid overuse.

As exemplified, FIG. 1A illustrates the power of VLP display in the present invention by showing a comparison of the IgG titers elicited in response to a peptide when displayed on Qbeta VLPs vs. KLH. In particular, a single dose of the VLP vaccine elicits anti-peptide antibodies as early as a week after immunization, and high titer responses by 2-3 weeks. In contrast, antibody responses to the KLH-conjugated peptide are slower and reach lower titers. Moreover, VLPs elicit long-lived antibody responses. In FIG. 1B, the inventors show that a single immunization with a VLP-based vaccine leads to antibodies that last, essentially, for the lifetime of the subject (mouse). Previous approaches for generating antibodies against opioids largely relied on attaching the drug to a carrier protein, such as KLH or Tetanus Toxoid, to provide a source of linked T-cell epitopes (1, first set of references). Those vaccine strategies require the addition of exogenous adjuvants and multiple boosts in order to effectively elicit antibodies. The inventors propose an alternative approach, using Qbeta or AP205 VLPs, to elicit high-titer and long-lasting antibodies more rapidly, potentially after only one dose, and without the use of exogenous adjuvants. Notably, there is a well-established clinical pathway for bacteriophage VLP-based vaccines. As of late 2017, at least seven Qbeta bacteriophage VLP-based vaccines had entered clinical trials, including several that had moved into phase II/IIa trials (16). These include VLP-based vaccines for Alzheimer's Disease, allergy, and hypertension that target amyloid-beta (17), dust mite allergen (18), and angiotensin II (19), respectively. These trials also demonstrated that VLP-based vaccines are highly immunogenic in humans.

For these reasons, VLPs can be used as a platform to elicit rapid, high titer, and long-lasting antibody responses to opioids. These features are required for effective vaccine-based treatment for opioid use disorder. The present vaccines provide an unexpectedly quick immunogenic response to the vaccines of the present invention which represents an unexpected result.

In an embodiment, the present invention is directed to a composition comprising: (a) a virus-like particle (VLP) comprising a bacteriophage coat protein: and (b) at least one conjugated opiate determinant; wherein said opioid determinant is displayed on said virus-like particle, and wherein said determinant comprises a conjugated opiate derived from an opioid compound. In embodiments, the opiate is conjugated to the surface of the VLP at high density. In an embodiment, the opiate conjugate determinant is displayed at one or more lysine residues at the A-B loop, N-terminus or carboxy terminus of said bacteriophage coat protein.

In embodiments, the bacteriophage coat protein used to form the VLPs is a coat protein derived from Qbeta or AP205 bacteriophage, preferably a coat protein derived from Qbeta bacteriophage.

In embodiments, in the composition according to the present invention the opiate conjugate determinant is displayed at one or more nucleophilic or electrophilic amino acid residues on the surface of the bacteriophage, preferably at a plurality of lysine residues on the surface of the VLP. In embodiments, the opiate conjugate is displayed on the bacteriophage at the lysine residues by covalently binding an opioid molecule to the lysine residues through a linker group. In embodiments, the linker group comprises a 4 to 15 mer, preferably a 4 to 10 mer oligopeptide covalently bonded to a crosslinker as described herein.

In embodiments of the present invention, the oligopeptide of the opiate conjugate is covalently bonded to an electrophilic or nucleophilic group of the opioid molecule (e.g. a carbonyl group, a vinyl group, an amine or hydroxyl group which optionally has been modified to facilitate the binding of the oligopeptide to the opioid molecule as depicted in FIG. 2 or FIG. 7) and the crosslinker is bonded to the nucleophilic or electrophilic amino acid residues, preferably lysine residues on the surface of the bacteriophage through the crosslinker. In embodiments, the oligopeptide of the linker is a 4 to 15 mer, preferably a 4 to 10 mer oligopeptide comprising neutral amino acid residues bonded to nucleophilic electrophilic sites, often an amine or hydroxyl group on the opioid molecule. In embodiments, on one end of the oligopeptide, often the carboxyl terminus, the oligopeptide comprises a cysteinyl group or other amino acid which may be used to link the oligopeptide to the crosslinker. The amino end of the oligopeptide may optionally be conjugated to the opioid molecule through the use of a short amide linker (e.g. a C₁-C₄ alkyl amide group which forms a urea or urethane group with the opioid radical) or a phenol group, among others.

In embodiments, the neutral amino acid residues are selected from the group consisting of glycine, alanine, valine, leucine, isoleucine, phenylalanine, tryptophan, methione, proline, serine and mixtures thereof. Embodiments, the neutral amino acids often are selected from the group consisting of glycine, serine and mixtures thereof, more often glycine.

In embodiments, the opiate conjugate comprises an opiate radical selected from the group consisting of a radical of codeine, fentanyl, hydrocodone, hydromorphone, meperidine, methadone, morphine, oxycodone, diacetylmorphine (heroin), hydroxylmorphine, 2,4-dinitrophenylmorphine, 6-methyldihydromorphine, 6-methylenedihydrodesoxymorphine, 6-acetyldihydromorphine, chloranaltrexamine, chloroxymorphamine, dexomorphine, dihydromorphine, ethyldihydromorphine, hydromorphinol, methyldesorphine, morphine methyl bromide, n-phenylnordesomorphine, N-phenylnormorphine, 6-nicotinoyldihydromorphine, acetylpropionylmorphine, 3,6-dibutanoylmorphine, dibutyrylmorphine, dibenzoylmorphine, diformylmorphine, diacetyl morphine (herone), dipropanooymorphine, nicomorphine, 6-monoacetylecodeine, benzylmorphine, codeine methylbromide, desocodeine, dimethylmorphine, ethyldihydromorphine, heterocodeine, dihydrocodeine, isocodeine, morpholinylethylmorphine, myrophine, transisocodeine, acetylcodone, acetylmorphone, dihydrocodeine, hydroxycodeine, codeinone, hydrocodone, hydromorphone, morphinol, morphinone and mixtures thereof. In embodiments, the opiate conjugate comprises an opiate radical selected from the group consisting of a radical of codeine, fentanyl, hydrocodone, hydromorphone, meperidine, methadone, morphine, oxycodone and diacetylmorphine (heroin), often fentanyl, heroin, morphine, 6-acetylmorphine, oxycodone or hydrocodone or fentanyl, heroin, morphine or oxycodone.

In embodiments, the present invention is directed to a population of virus-like particles as otherwise described herein.

In embodiments, the present invention is directed to a pharmaceutical composition comprising a population of virus-like particles as described herein in combination with a pharmaceutically acceptable carrier, additive and/or excipient. In embodiments, the composition is formulated for administration to a subject or patient as a vaccine. In embodiments the pharmaceutical composition or vaccine comprises an adjuvant (e.g., Advax, MF 59, CPG 1018, AS01B, AS03, AS04, etc.).

In embodiments, the present invention is directed to a method for enhancing an immune response against an opiate compound in a patient or subject in need comprising introducing a pharmaceutical composition comprising a population of VLPs as otherwise described herein to said subject or patient, wherein an enhanced immune response against said opiate compound is produced in said patient or subject. In embodiments, the present invention is directed to a method for reducing the likelihood of drug-induced antinociception in a patient or subject in need. In embodiments, the present invention is directed to a method wherein the composition is prophylactic for an opiate induced disorder.

In embodiments, the present invention is direct to a method of inducing an immunogenic response in a patient or subject comprising administering a composition comprising an effective amount of a population of opiate conjugated VLPs as otherwise described herein to said patient or subject.

In embodiments, the present invention is directed to a method for treating or inhibiting opioid use disorder or a symptom thereof in a patient or subject in need comprising administering to said patient or subject a composition comprising an effective amount of a population of opiate conjugated VLPs as otherwise described herein to said patient or subject.

In embodiments, the disorder is opiate dependency. In embodiments, the symptom is nausea, vomiting, weakened immune system, slow breathing rate/respiratory depression, coma, increased risk of infectious disease including hepatitis, hallucinations, collapsed veins or clogged blood vessels, risk of choking, increased tolerance to opioids, an inability to stop or reduce usage, narcolepsy, extreme weight loss or gain, anxiety, sweating, insomnia, agitation, tremors, muscle aches, nausea, vomiting, diarrhea or extreme mental and physical discomfort.

In embodiments, the present invention is directed to a method for treating or reducing the likelihood of an opioid overdose or a symptom thereof in a patient or subject in need comprising administering to said patient a composition comprising an effective amount of a population of opiate conjugated VLPs as otherwise described herein to said patient or subject.

In embodiments, the symptom is nausea, vomiting, diarrhea, slow breathing rate/respiratory depression, coma, hallucinations, collapsed veins or clogged blood vessels, risk of choking, narcolepsy, sweating, insomnia, agitation, tremors, muscle aches, nausea, vomiting, diarrhea or extreme mental and physical discomfort.

The present invention is therefore directed to vaccines which target opioid drugs including fentanyl, heroin (including its active metabolites morphine and 6-acetylmorphine), morphine, oxycodone, and hydrocodone and other opioid drugs as otherwise disclosed herein for prophylactic and/or therapeutic purposes.

### BRIEF DESCRIPTION OF THE FIGURES

FIG. 1 shows that Bacteriophage VLPs elicit high titer, long-lasting antibodies in a single dose. (A) A peptide of interest was conjugated to Qbeta VLPs using SMPH bifunctional crosslinker (blue) or KLH (red and pink). Groups of mice (n=6) were immunized intramuscular with QB-PEP (5 ug), KLH-PEP (15 ug) or KLH-PEP (25 ug). Serum was collected at 1-, 2-, and 3-weeks post immunization and assessed for PEP-specific IgG by ELISA. (B) Mice (n=5) were immunized intramuscularly with 1 or 3 doses with bacteriophage VLPs displaying a peptide representing a Human Papillomavirus neutralizing epitope, and peptide-specific IgG titers were measured by ELISA for nearly 2-years post-vaccination.

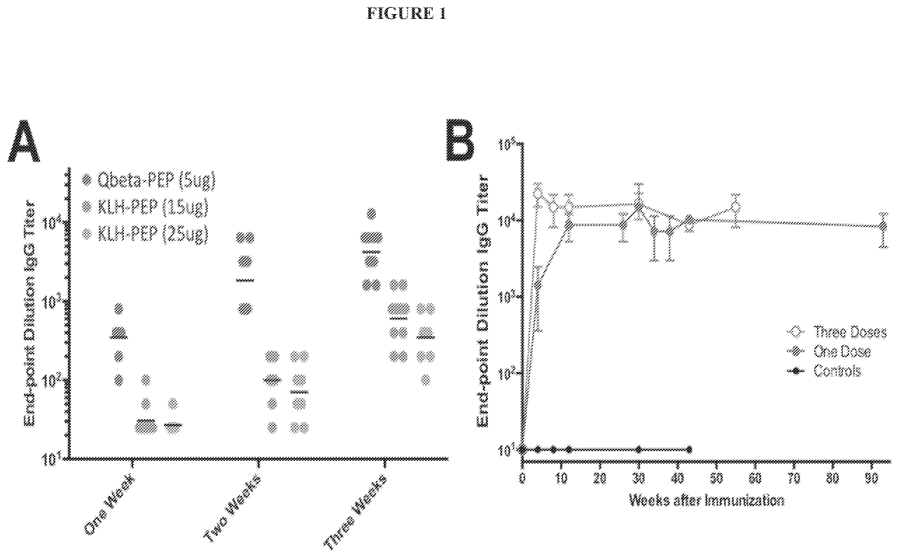

FIG. 2 shows the chemical synthesis of opioid drug derivatives for conjugation with peptide linker and chemical conjugation to Qβ-VLPs. Oxycodone, morphine, and 6-acetylmorphine were synthesized to include a (Gly)₄Cys peptide at the indicated active site. (B) Qβ VLP structure with surface-exposed lysines shown in yellow. (A) Scheme for conjugation of drug-peptide to Qβ VLPs (oxycodone shown). (C) Coomassie-stained SDS-PAGE gels successful conjugation of drug-peptide to Qβ-VLPs.

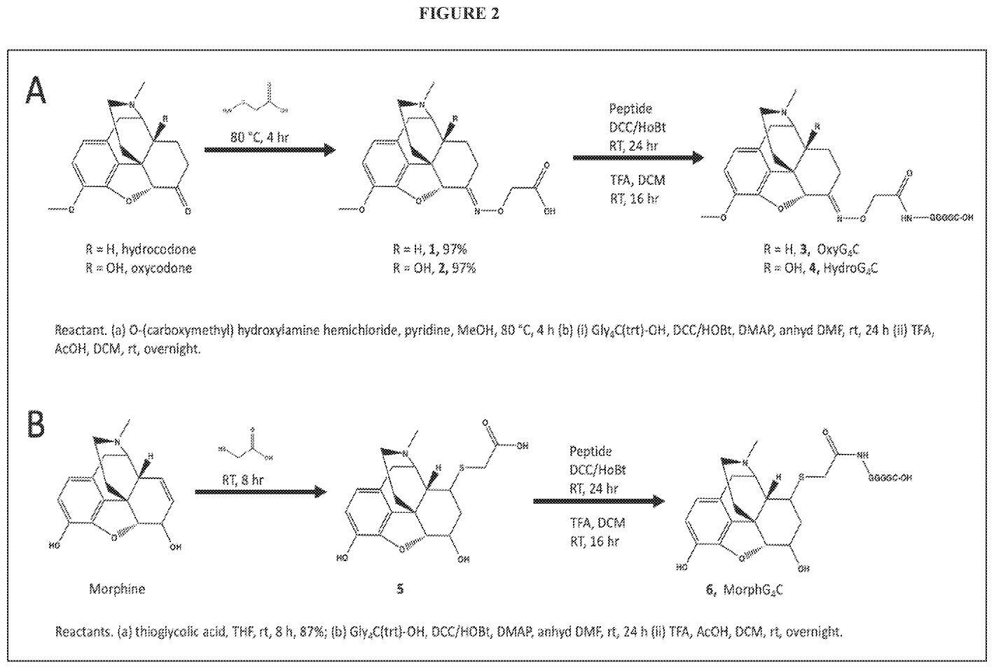

FIG. 3 shows the chemical conjugation of peptides to Qbeta VLPs with SMPH bifunctional crosslinker. (A) Schematic showing the strategy for conjugation of opioid-(Gly)₄-Cys to Qbeta VLPs. Surface-exposed lysines (yellow space fill) on Qβ VLP and cysteine (—SH sidechain) (red) are available for SMPH conjugation. (C) Qbeta VLP with surface-exposed lysines (yellow space fill) indicated. (D) Example of a successful conjugation of a sulfhydryl-bearing peptide to Qbeta VLP. Each Coomassie-stained band represents a different coat-protein species containing 1, 2, 3, or 4 peptides per coat protein.

FIG. 4 shows an Immunization schedule for the methods conducted pursuant to the present invention.

FIG. 5 shows a timeline for conducting experiments according to the present invention.

FIG. 6 shows that Qβ-oxycodone vaccine elicits high titer antibodies within 7 days post immunization and protects against lethal oxycodone overdose. Mice (N=5) were intramuscularly immunized one time with 20 ug of Qβ-oxycodone, Qβ-VLP alone (control), KLH-oxycodone, KLH alone (control), or PBS (control) or Qβ-morphine. (A) At various times post immunization, sera were collected and assessed for anti-oxycodone or anti-morphine IgG by ELISA. (B) In a separate experiment, vaccinated mice (N=6) were challenged with a lethal dose of oxycodone (426 mg/kg body weight) at 20 days post-immunization and monitored for survival for 24 hours.

FIG. 7 shows exemplary opioid haptens for use in the present invention.

FIG. 8 shows an early phase of vaccine development.

FIG. 9 shows a timeline for phase 1 of vaccine development.

FIG. 10 shows that a dose of Qβ-OXY immunogen impacts longevity of IgG response. Mice were immunized once with various doses of Qβ-OXY and serum was assessed for anti-oxycodone IgG by ELISA at various times post-immunization.

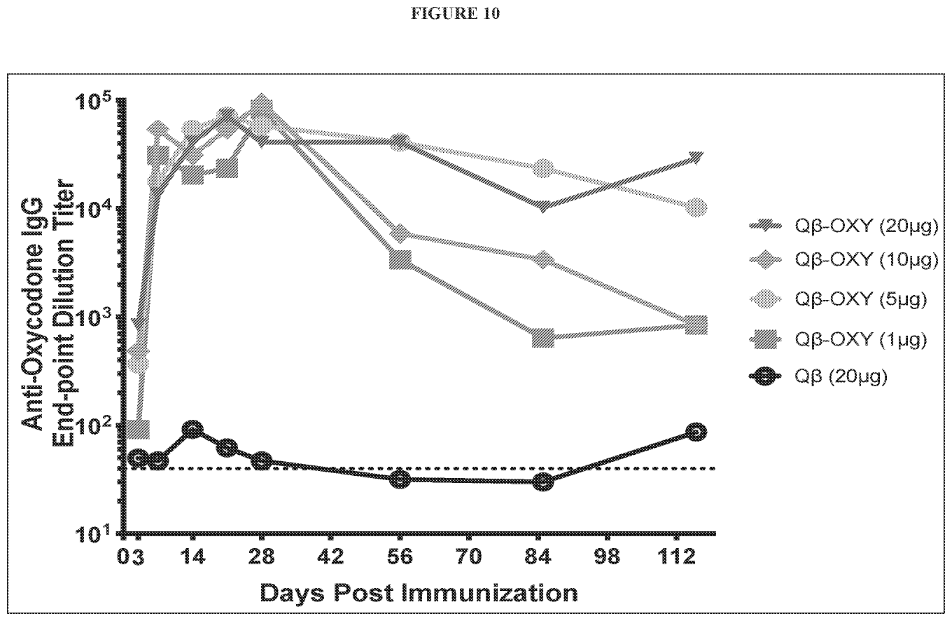

FIG. 11 shows (A) Consistent pattern of respiratory depression with 75 μg/kg iv infusion of fentanyl. (B) Higher doses of fentanyl create similar patterns of acute depression, with 300 μg/kg iv infusion causing more pronounced sustained depression.

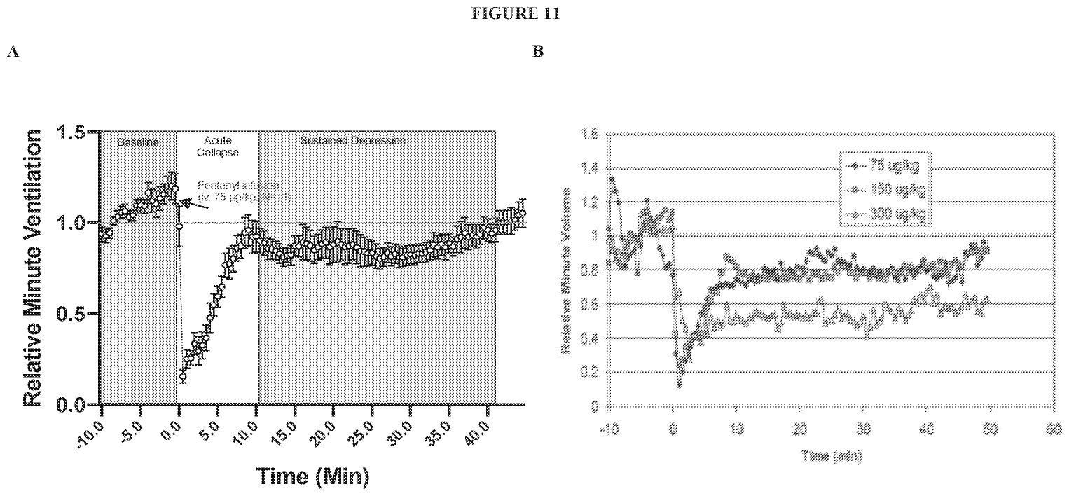

FIG. 12 shows a timeline for phase 2 vaccine development.

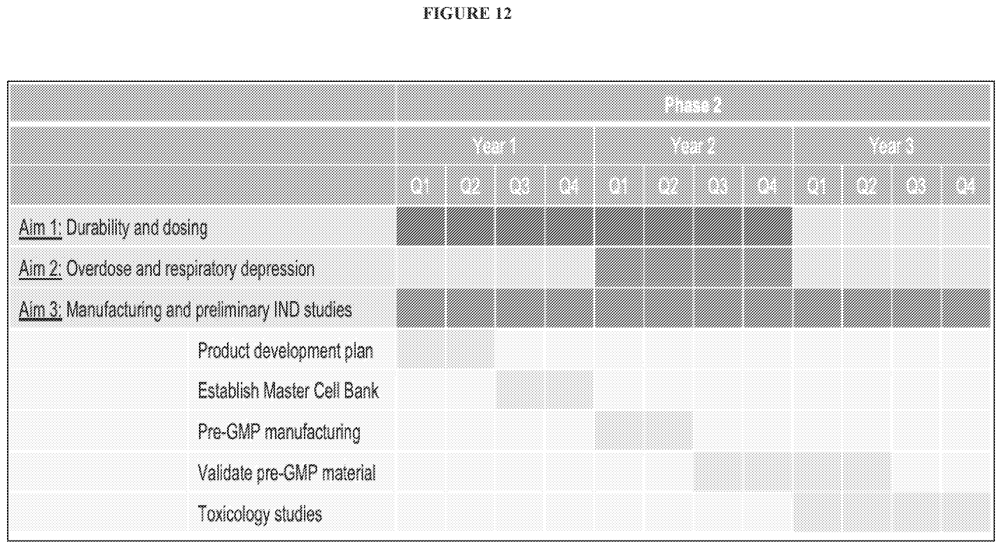

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”: IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal and preferably a human, to whom treatment, including prophylactic treatment (prophylaxis), with the immunogenic compositions and/or vaccines according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe a number of VLP's or an amount of a VLP-containing composition which, in context, is used to produce or effect an intended result, whether that result relates to the prophylaxis and/or therapy of opiate dependency and/or opioid overdose as otherwise described herein. The term effective subsumes all other effective amount or effective concentration terms (including the term “therapeutically effective”) which are otherwise described or used in the present application.

As used herein, the term “opioid”, “opiate” or “opioid drug” is used to describe a compound which interacts with an opiate receptor to produce a pharmacological response, often analgesia. These agents typically produce dependency and related symptoms among other symptoms as described herein often occur when the drug is no longer administered. These drugs are chemically conjugated with VLPs according to the present invention in order to produce an immunogenic response in a subject administered same against opiates as otherwise described herein. Typical opiates include, for example, codeine, fentanyl, hydrocodone, hydromorphone, meperidine, methadone, morphine, oxycodone and diacetylmorphine (heroin), hydroxylmorphine, 2,4-dinitrophenylmorphine, 6-methyldihydromorphine, 6-methylenedihydrodesoxymorphine, 6-acetyldihydromorphine, chloranaltrexamine, chloroxymorphamine, dexomorphine, dihydromorphine, ethyldihydromorphine, hydromorphinol, methyldesorphine, morphine methyl bromide, n-phenylnordesomorphine, N-phenylnormorphine, 6-nicotinoyldihydromorphine, acetylpropionylmorphine, 3,6-dibutanoylmorphine, dibutyrylmorphine, dibenzoylmorphine, diformylmorphine, diacetyl morphine (herone), dipropanooymorphine, nicomorphine, 6-monoacetylecodeine, benzylmorphine, codeine methylbromide, desocodeine, dimethylmorphine, ethyldihydromorphine, heterocodeine, dihydrocodeine, isocodeine, morpholinylethylmorphine, myrophine, transisocodeine, acetylcodone, acetylmorphone, dihydrocodeine, hydroxycodeine, codeinone, hydrocodone, hydromorphone, morphinol and morphinone, among others.

Preferred opioid compounds for use in the present invention as conjugates to VLPs include codeine, fentanyl, hydrocodone, hydromorphone, meperidine, methadone, morphine, oxycodone and diacetylmorphine (heroin).

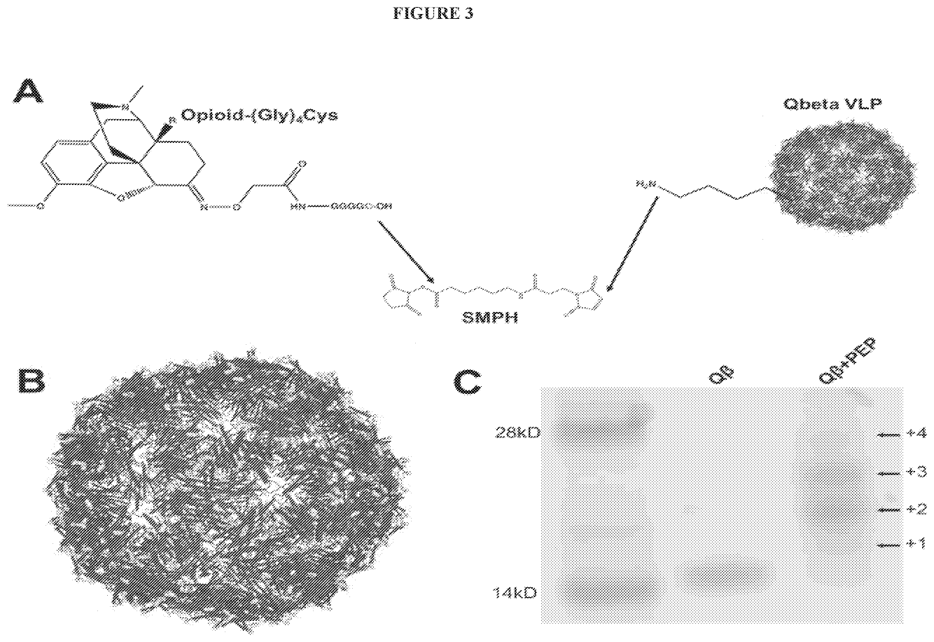

The term “opiate conjugate” refers to an opioid molecule which is conjugated to the external surface of a VLP, often a Qβ or AP205 bacteriophage, often a Qβ bacteriophage through a linker molecule to a nucleophilic amino acid on the surface of the bacteriophage. In embodiments, the nucleophilic amino acid is a lysine residue on the surface of the bacteriophage. The opioid is conjugated to the bacteriophage through a linker molecule. Often the linker molecule comprises a 4-15 mer, often a 4-12 mer, a 4-10 mer, a 4-8 mer a 4-6 mer or a 4 mer oligopeptide (preferably comprising neutral amide acid residues) which is covalently bonded to a crosslinker molecule as described herein to form the linker. The oligonucleotide is covalently linked at one end to the opioid radical hapten often through and electrophilic or nucleophilic functional group on the opioid (often a carboxyl group, a vinyl group, an amine group or a hydroxyl group, more often an amine group which is optionally further linked by an amide or other group, often a short, C₁-C₄ alkyl amide) and on the other end to the crosslinker, which links the VLP to the oligopeptide and the opioid hapten. This is shown in FIG. 3, on the linker molecule the linker which crosslinks the oligonucleotide which is covalently bonded to the opioid radical hapten covalently bonded to the oligonucleotide to the opioid radical hapten.

The term “crosslinker” or “crosslinking agent” refers to a chemical compound used to covalently bind, or conjugate, biomolecules together, such as an oligopeptide to a VLP or an oligopeptide to an opioid hapten. The term “protein crosslinking” refers to utilizing protein crosslinkers to conjugate peptides or proteins together. Crosslinking agents for use herein possess reactive moieties specific to various electrophilic or nucleophilic functional groups (e.g., sulfhydryls, amines, carbohydrates, carboxyl groups, hydroxyl groups, carbonyls, etc.) on proteins, peptides, or other molecular complexes or molecules such as opioids as described herein. The atoms separating a crosslinker agent's reactive groups, and eventually the conjugated oligopeptide/VLP or oligopeptide/opioid form the “spacer arm”. A zero-length crosslinker refers to protein crosslinkers that join two molecules without adding additional spacer arm atoms. Homobifunctional crosslinker reagents have the same reactive group on both ends of the spacer arm (i.e., Amine Reactive-Amine Reactive); while heterobifunctional crosslinkers have different reactive groups on each end of a spacer arm (i.e., Sulfhydryl Reactive-Amine Reactive). It is noted that in addition to the following crosslinking agents, additional short-chain crosslinking agents such as short-chain alkyl amides (CH₂)ᵢC(O)NH₂, (CH₂)ᵢC(O), C(O)(CH₂)ᵢC(O), NHC(O)(CH₂)ᵢC(O) or NHC(O)(CH₂)ᵢC(O)NH groups where i is from 1 to 4, can be used to link an opioid hapten to an oligopeptide or a crosslinker to a lysine group on the VLP. The following crosslinking agents are exemplary for use in the present invention:

ANB-NOS (N-5-Azido-2-nitrobenzoyloxysuccinimide)
BMPS N-(β-Maleimidopropyloxy)succinimide ester
EMCS (N-[e-Maleimidocaproyloxy]succinimide ester)

### GMBS (N-[Gamma-Maleimidobutyryloxy] Succinimide)

LC-SPDP Succinimidyl 6-(3-[2-pyridyldithio]-propionamido)hexanoate)
MBS (m-Maleimidobenzoyl-N-hydroxysuccinimide ester)
PDPH (3-[2-Pyridyldithio]propionyl hydrazide)
SBA (N-Succinimidyl bromoacetate)
SIA (N-Succinimidyl iodoacetate)
Sulfo-SIA N-Sulfosuccinimidyl iodoacetate)
SMCC (Succinimidyl-4-[N-maleimidomethyl]cyclohexane-1-carboxylate)
SMPB (N-Succinimidyl 4-[4-maleimidophenyl]butyrate)
SMPH (Succinimidyl-6-[β-maleimidopropionamido]hexanoate)
SPDP (N-Succinimidyl 3-[2-pyridyldithio]-propionate)
Sulfo-LC-SPDP Sulfosuccinimidyl 6-(3′-[2-pyridyldithio]-propionamido)hexanoate
Sulfo-MBS (m-Maleimidobenzoyl-N-hydroxysulfosuccinimide ester)
Sulfo-SANPAH (N-Sulfosuccinimidyl-6-[4′-azido-2′-nitrophenylamino]hexanoate)
sulfo-SMCC (Sulfosuccinimidyl-4-[N-maleimidomethyl]cyclohexane-1-carboxylate)
BS2G (Bis[Sulfosuccinimidyl] glutarate)
BS3 (Bis[sulfosuccinimidyl] suberate)
DSG (Disuccinimidyl glutarate)
DSP (Dithiobis[succinimidyl propionate])
DSS (Disuccinimidyl suberate)
DSSeb (Disuccinimidyl sebacate)
DST (Disuccinimidyl tartrate)

### DTSSP (3,3′-Dithiobis[sulfosuccinimidylpropionate])

EGS (Ethylene glycolbis(succinimidylsuccinate)
Sulfo-EGS Ethylene glycolbis(sulfosuccinimidylsuccinate)

### CDI (N,N′-Carbonyldimidazole)

DCC (N,N′-dicyclohexylcarbodiimide)
EDC-HCl 1-Ethyl-3-[3-dimethylaminopropyl]carbodiimide hydrochloride)

### NHS (N-hydroxysuccinimide) and

### Sulfo-NHS (N-hydroxysulfosuccinimide)

Preferred crosslinkers for use in the present invention are heterobifunctional agents which are capable of linking Amine-to-Sulfhydryl groups. Exemplary crosslinking agents include:

SIA (succinimidyl iodoacetate)
SBAP (succinimidyl 3-(bromoacetamido)propionate)
SIAB (succinimidyl (4-iodoacetyl)aminobenzoate)
Sulfo-SIAB (sulfosuccinimidyl (4-iodoacetyl)aminobenzoate)
AMAS (N-α-maleimidoacet-oxysuccinimide ester)
BMPS (N-β-maleimidopropyl-oxysuccinimide ester)
GMBS (N-γ-maleimidobutyryl-oxysuccinimide ester)
MBS (m-maleimidobenzoyl-N-hydroxysuccinimide ester)
SMCC (succinimidyl 4-(N-maleimidomethyl)cyclohexane-1-carboxylate)
EMCS (N-ε-malemidocaproyl-oxysuccinimide ester)
Sulfo-GMBS (N-γ-maleimidobutyryl-oxysulfosuccinimide ester)
Sulfo-MBS (m-maleimidobenzoyl-N-hydroxysulfosuccinimide ester)
Sulfo-SMCC (sulfosuccinimidyl 4-(N-maleimidomethyl)cyclohexane-1-carboxylate)
Sulfo-EMCS (N-ε-maleimidocaproyl-oxysulfosuccinimide ester)
Sulfo-SMPB (sulfosuccinimidyl 4-(N-maleimidophenyl)butyrate)
SMPB (succinimidyl 4-(p-maleimidophenyl)butyrate)
SMPH (Succinimidyl 6-((beta-maleimidopropionamido)hexanoate))
LC-SMCC (succinimidyl 4-(N-maleimidomethyl)cyclohexane-1-carboxy-(6-amidocaproate)) and
Sulfo-KMUS (N-κ-maleimidoundecanoyl-oxysulfosuccinimide ester).

As used herein, the term “polynucleotide” refers to a polymeric form of nucleotides of any length, either ribonucleotides or deoxynucleotides, and includes both double- and single-stranded DNA and RNA. A polynucleotide may include nucleotide sequences having different functions, such as coding regions, and non-coding regions such as regulatory sequences (e.g., promoters or transcriptional terminators). A polynucleotide can be obtained directly from a natural source, or can be prepared with the aid of recombinant, enzymatic, or chemical techniques. A polynucleotide can be linear or circular in topology. A polynucleotide can be, for example, a portion of a vector, such as an expression or cloning vector, or a fragment.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e g., dimers, tetramers). Thus, the terms peptide, oligopeptide, and protein are all included within the definition of polypeptide and these terms are used interchangeably. It should be understood that these terms do not connote a specific length of a polymer of amino acids, nor are they intended to imply or distinguish whether the polypeptide is produced using recombinant techniques, chemical or enzymatic synthesis, or is naturally occurring.

The term “single-chain dimer” refers to a normally dimeric protein whose two subunits of coat polypeptide of a RNA bacteriophage have been genetically (chemically, through covalent bonds) fused into a single polypeptide chain. Specifically, in the present invention single-chain dimer versions of bacteriophage, preferably Qbeta coat proteins are constructed. Each of these proteins is naturally a dimer of identical polypeptide chains. In certain of the bacteriophages coat protein dimers the N-terminus of one subunit lies in close physical proximity to the C-terminus of the companion subunit. Single-chain coat protein dimers were produced using recombinant DNA methods by duplicating the DNA coding sequence of the coat proteins and then fusing them to one another in tail to head fashion. The result is a single polypeptide chain in which the coat protein amino acid appears twice, with the C-terminus of the upstream copy covalently fused to the N-terminus of the downstream copy. Normally (wild-type) the two subunits are associated only through noncovalent interactions between the two chains. In the single-chain dimer these noncovalent interactions are maintained, but the two subunits have additionally been covalently tethered to one another. This greatly stabilizes the folded structure of the protein and confers to it its high tolerance of peptide insertions as described above.

This application makes frequent reference to coat protein's “AB-loop”. The RNA phage coat proteins possess a conserved tertiary structure. Opioid compounds conjugated into the AB-loop, or at the N- or C-terminus are exposed on the surface of the VLP and are strongly immunogenic.

The amino acid residues described herein are preferred to be in the “L” isomeric form. However, residues in the “D” isomeric form can be substituted for any L-amino acid residue, as long as the desired functional is retained by the polypeptide. NH₂ refers to the free amino group present at the amino terminus of a polypeptide. COOH refers to the free carboxy group present at the carboxy terminus of a polypeptide.

The term “valency” is used to describe the density of the opioid conjugates displayed on VLPs according to the present invention. Valency in the present invention may range from low valency to high valency (“high density”), from less than 1 to more than about 180, preferably 90 to 180 or more (e.g between 180-720, or between 1 and 4 conjugates per coat protein in the VLP). Immunogenic compositions according to the present invention comprise VLPs which are preferably high valency and comprise VLPs which display at least 50-60 up to about 180 or more, often 90-720 or more, often 180 to 720 or more crosslinked conjugated opioids per VLP as otherwise described herein. In embodiments, at least 90 opioid conjugates are “high density” because the display of 90 copies of antigen/hapten on the surface of the VLP produces high titer antibodies.

The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5-end of the mRNA and a transcription terminator sequence located just downstream of the open reading frame at the 3′-end of the mRNA. A coding sequence can include, but is not limited to, DNA, cDNA, and recombinant nucleic acid sequences.

A “heterologous” region of a recombinant cell is an identifiable segment of nucleic acid within a larger nucleic acid molecule that is not found in association with the larger molecule in nature.

An “origin of replication” refers to those DNA sequences that participate in DNA synthesis.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

In bacteria, transcription normally terminates at specific transcription termination sequences, which typically are categorized as rho-dependent and rho-independent (or intrinsic) terminators, depending on whether they require the action of the bacterial rho-factor for their activity. These terminators specify the sites at which RNA polymerase is caused to stop its transcription activity, and thus they largely define the 3′-ends of the RNAs, although sometimes subsequent action of ribonucleases further trims the RNA.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

An “antibiotic resistance gene” refers to a gene that encodes a protein that renders a bacterium resistant to a given antibiotic. For example, the kanamycin resistance gene directs the synthesis of a phosphotransferase that modifies and inactivates the drug. The presence on plasmids of a kanamycin resistance gene provides a mechanism to select for the presence of the plasmid within transformed bacteria. Similarly, the chloramphenicol resistance gene allows bacteria to grow in the presence of the drug by producing an acetyltransferase enzyme that inactivates the antibiotic through acetylation.

The term “PCR” refers to the polymerase chain reaction, a technique used for the amplification of specific DNA sequences in vitro. The term “PCR primer” refers to DNA sequences (usually synthetic oligonucleotides) able to anneal to a target DNA, thus allowing a DNA polymerase (e.g. Taq DNA polymerase) to initiate DNA synthesis. Pairs of PCR primers are used in the polymerase chain reaction to initiate DNA synthesis on each of the two strands of a DNA and to thus amplify the DNA segment between two primers. Representative PCR primers which used in the present invention are those which are presented in the examples section hereof.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid, which normally replicate independently of the bacterial chromosome by virtue of the presence on the plasmid of a replication origin. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

It should be appreciated that also within the scope of the present invention are nucleic acid sequences encoding the polypeptide(s) of the present invention, which code for a polypeptide having the same amino acid sequence as the sequences disclosed herein, but which are degenerate to the nucleic acids disclosed herein. By “degenerate to” is meant that a different three-letter codon is used to specify a particular amino acid.

As used herein, “epitope” refers to an antigenic determinant of a polypeptide. An epitope could comprise 3 amino acids in a spatial conformation which is unique to the epitope. Generally an epitope consists of at least 4 such amino acids, and more often, consists of at least 5-10 such amino acids. Methods of determining the spatial conformation of amino acids are known in the art, and include, for example, x-ray crystallography and 2-dimensional nuclear magnetic resonance.

As used herein, the term “coat protein(s)” refers to the protein(s) of a bacteriophage or a RNA-phage capable of being incorporated within the capsid assembly of the bacteriophage or the RNA-phage. These include, but are not limited to Qβ, AP205, PP7, MS2, AP205, R17, SP, PP7, GA, M11, MX1, f4, Cb5, Cb12r, Cb23r, 7s and f2 RNA bacteriophages. Preferred coat proteins which are used in the present invention include coat proteins from bacteriophages include Qβ, AP205, PP7 and MS2. Preferably, Qβ or AP205, most often Qβ coat polypeptides are used to create conjugated VLPs according to the present invention.

As used herein, a “coat polypeptide” as defined herein is a polypeptide of the full length coat protein of the bacteriophage, a polypeptide fragment of the coat protein that possesses coat protein function and additionally encompasses the full length coat protein as well or single-chain variants thereof.

As used herein, the term “immune response” refers to a humoral immune response and/or cellular immune response leading to the activation or proliferation of B- and/or T-lymphocytes and/or antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic opiate” is a conjugated opiate that elicits a cellular and/or humoral immune response as described above, whether alone or linked to a carrier in the presence or absence of an adjuvant. Preferably, antigen presenting cell may be activated.

As used herein, the term “vaccine” refers to a formulation which contains the composition of the present invention and which is in a form that is capable of being administered to an animal, often a human patient or subject.

As used herein, the term “virus-like particle of a bacteriophage” refers to a virus-like particle (VLP) resembling the structure of a bacteriophage, being non-replicative and noninfectious, and lacking at least the gene or genes encoding for the replication machinery of the bacteriophage, and typically also lacking the gene or genes encoding the protein or proteins responsible for viral attachment to or entry into the host.

This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA bacteriophage coat protein: The capsid structure formed from the self-assembly of one or more subunits of RNA bacteriophage coat protein and optionally containing host RNA is referred to as a “VLP of RNA bacteriophage coat protein”. In a particular embodiment, the capsid structure is formed from the self assembly of 90 coat protein single-chain dimers or 180 coat protein monomers. In the case of Qβ or AP205 VLPs 180 coat protein monomers typically self-assemble into the VLP.

A nucleic acid molecule is “operatively linked” to, or “operably associated with”, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

### Opioid Dependency, Immunogenicity, and Prophylactic Efficacy

“Opioid dependency” or “Opioid Overdose” includes, but is not limited to, the disorders identified in this application which are caused by opioid use. Immunogenicity and prophylactic efficacy (e.g. whether a composition is prophylactic for opioid induced disorders such as dependency or overdose) may be evaluated either by the techniques and standards mentioned in this section, or through other methodologies that are well-known to those of ordinary skill in the art.

To assess immunogenicity (e.g. whether a composition has induced a high titer antibody responses against opiates), an anti-opiate geometric mean titer (GMT) can be measured by ELISA, e.g. after a few weeks of treatment (e.g. 3 or 4 weeks) and after administration of a few dosages (e.g. 3 or 4). The percentage of subjects who seroconverted for opiate antigenicity (OA) after a few weeks of treatment (e.g. 3 or 4 weeks) and after administration of a few dosages (e.g. 3 or 4) can also be determined to assess immunogenicity.

To assess efficacy assessment of sequestration of opiates in the blood (not getting to site of action in the brain) and the ability of the vaccine to block the analgesic effects of opiates is performed.

### Production of Virus-Like Particles

The present invention is directed to virus-like phage particles as well as methods for producing these particles in vivo as well as in vitro. As used herein, producing virions “in vitro” refers to producing virions outside of a cell, for instance, in a cell-free system, while producing virions “in vivo” refers to producing virions inside a cell, for instance, an Escherichia coli or Pseudomonas aeruginosa cell or a yeast cell among others.

### Bacteriophages

The VLPs described here consist of assemblies of the coat proteins of single-strand RNA bacteriophage [RNA Bacteriophages, in The Bacteriophages. Calendar, R L, ed. Oxford University Press. 2005]. The known viruses of this group attack bacteria as diverse as E. coli, Pseudomonas and Acinetobacter. Each possesses a highly similar genome organization, replication strategy, and virion structure. In particular, the bacteriophages contain a single-stranded (+)-sense RNA genome, contain maturase, coat and replicase genes, and have small (<300 angstrom) icosahedral capsids. These include but are not limited to Qβ, AP205, PP7, MS2, R17, SP, PP7, GA, M11, MX1, f4, Cb5, Cb12r, Cb23r, 7s and f2 RNA bacteriophages. Qβ and AP205 RNA bacteriophages are preferred, Qβ is most preferred. Qβ and AP205 RNA bacteriophages form self-assembled VLPs from 180 monomeric coat polypeptide units. Methods for producing these coat polypeptides are well known in the art. See, for example Freivalds, et al., J Biotechnol., 2006 May 29:123(3):297-303.

The information required for assembly of the icosahedral capsid shell of this family of bacteriophage is contained entirely within coat protein itself. For example, purified coat protein can form capsids in vitro in a process stimulated by the presence of RNA [Beckett et al., 1988, J. Mol Biol 204: 939-47]. Moreover, coat protein expressed in cells from a plasmid assembles into a virus-like particle in vivo [Peabody, D. S., 1990, J Biol Chem 265: 5684-5689].

The preferred VLP for use in the present invention is a Qbeta or Qβ VLP. These VLPs are typically made by transformation of E. coli with a plasmid expressing the Qbeta coat protein as a monomer under a lac promoter. Colonies are selected on kanamycin Luria Broth (LB) agar plates. A single colony is used to inoculate LB broth and grown overnight at 37 degrees C. This is then used to inoculate a larger culture. Cultures are shaken at 37 degrees C. for several hours until OD600 reaches 0.8. Then the expression of the Qbeta coat protein is induced with Isopropyl β-d-1-thiogalactopyranoside (IPTG) and incubated another 3 hours. Then cells are pelleted and frozen at −20 degrees C. Lysis of bacteria is then performed in isotonic buffer with sonication. VLPs are isolated by size exclusion chromatography and endotoxin is depleted with sequential Triton-X-100 phase extraction. There are many alternative methods to isolate the VLPs, which are well known in the art.

### RNA Bacteriophage Coat Polypeptide

The coat polypeptides useful in the present invention also include those having similarity with one or more of the coat polypeptide described above. The similarity is referred to as structural similarity. Structural similarity may be determined by aligning the residues of the two amino acid sequences (i.e., a candidate amino acid sequence and the amino acid sequence) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate amino acid sequence can be isolated from a single stranded RNA virus, or can be produced using recombinant techniques, or chemically or enzymatically synthesized. Preferably, two amino acid sequences are compared using the BESTFIT algorithm in the GCG package (version 10.2, Madison Wis.), or the Blastp program of the BLAST 2 search algorithm, as described by Tatusova, et al. (FEMS Microbial Lett 1999, 174:247-250), and available at http://www.ncbi.nlm.nih.gov/blast/bl2seq/bl2.html. Preferably, the default values for all BLAST 2 search parameters are used, including matrix=BLOSUM62; open gap penalty=11, extension gap penalty=1, gap xdropoff=50, expect=10, wordsize=3, and optionally, filter on. In the comparison of two amino acid sequences using the BLAST search algorithm, structural similarity is referred to as “identities.” Preferably, a coat polypeptide also includes polypeptides with an amino acid sequence having at least 80% amino acid identity, at least 85% amino acid identity, at least 90% amino acid identity, or at least 95% amino acid identity to one or more of the amino acid sequences disclosed above. Preferably, a coat polypeptide is active. Whether a coat polypeptide is active can be determined by evaluating the ability of the polypeptide to form a capsid and package a single stranded RNA molecule. Such an evaluation can be done using an in vivo or in vitro system, and such methods are known in the art and routine. Alternatively, a polypeptide may be considered to be structurally similar if it has similar three-dimensional structure as the recited coat polypeptide and/or functional activity.

### The Opioid Conjugate

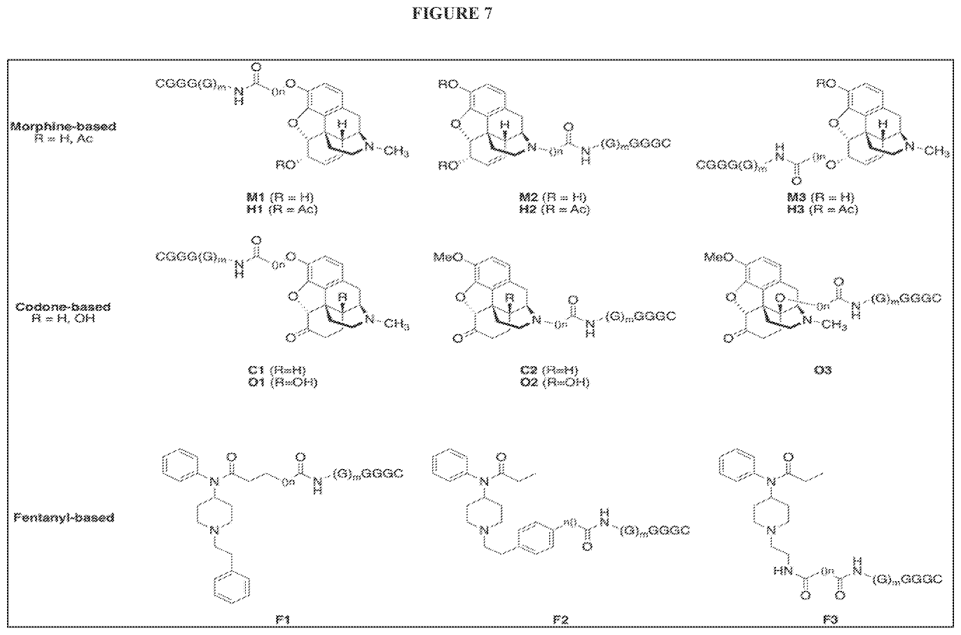

As described herein, in certain embodiments the opioid conjugate may be present (covalently linked) to the VLP in the A-B loop, at the N-terminus or the carboxy terminus of a coat polypeptide. Preferably, the opiate conjugate is covalently linked on the outer surface of the capsid. In particularly preferred embodiments, the approach is to attach the opiates to the lysines present on the surface of Qbeta VLPs. Attached FIGS. 3 and 7 shows this strategy.

### Qbeta LPs Lysine Positions:

The lysine residues which are available for conjugation on the coat polypeptide of the Qbeta VLPs of the present invention are set forth

| | |
|---|---|
| 1 | akletvtlgn igkdgkqtlv lnprgvnptn gvaslsqaga vpalekrvtv svsqpsrnrk |
| 61 | nykvqvkiqn ptactangsc dpsvtrqaya dvtfsftqys tdeerafvrt elaallaspl |
| 121 | lidaidqlnp ay |

Lysine amino acid residues are indicated above in bold. They are at amino acid positions 2, 13, 16, 46, 60, 63, and 67 of the monomeric coat polypeptide.

In preferred embodiments, the present invention is directed to A-B loop, N-terminal or C-terminal presentation of opiate conjugates on VLPs including PP7, MS2, AP205 and Qβ, preferably Qβ. These VLP-COs can be used singly or as a combination vaccine. The inventors show protection against opioid dependency using a vaccine consisting of a Qbeta VLP conjugated opioid as described herein.

In a particular embodiment, the coat polypeptide is a single-chain dimer containing an upstream and downstream subunit. Each subunit contains a functional coat polypeptide sequence. The opiate conjugate may be inserted in the upstream and/or downstream subunit at the sites mentioned herein above, e.g., the A-B loop, the N-terminus or a carboxyl erminus. In a particular embodiment, the coat polypeptide is a single chain dimer of a Qβ, PP7 or MS2 coat polypeptide, preferably a Qβ coat polypeptide, although a number of bacteriophage coat polypeptides may be used.

### Preparation of Transcription Unit

The transcription unit of the present invention comprises an expression regulatory region, (e.g., a promoter), a sequence encoding a coat polypeptide and transcription terminator. The RNA polynucleotide may optionally include a coat recognition site (also referred to a “packaging signal”, “translational operator sequence”, “coat recognition site”). Alternatively, the transcription unit may be free of the translational operator sequence. The promoter, coding region, transcription terminator, and, when present, the coat recognition site, are generally operably linked. “Operably linked” or “operably associated with” refer to a juxtaposition wherein the components so described are in a relationship permitting them to function in their intended manner. A regulatory sequence is “operably linked” to, or “operably associated with”, a coding region when it is joined in such a way that expression of the coding region is achieved under conditions compatible with the regulatory sequence. The coat recognition site, when present, may be at any location within the RNA polynucleotide provided it functions in the intended manner.

The invention is not limited by the use of any particular promoter, and a wide variety of promoters are known. The promoter used in the invention can be a constitutive or an inducible promoter. Preferred promoters are able to drive high levels of RNA encoded by me coding region encoding the coat polypeptide Examples of such promoters are known in the art and include, for instance, the lac promoter, T7, T3, and SP6 promoters.

The nucleotide sequences of the coding regions encoding coat polypeptides described herein are readily determined. These classes of nucleotide sequences are large but finite, and the nucleotide sequence of each member of the class can be readily determined by one skilled in the art by reference to the standard genetic code. Furthermore, the coding sequence of an RNA bacteriophage single chain coat polypeptide comprises a site for covalent binding of an opiate conjugate. In a particular embodiment, the site for insertion of the opiate conjugate is at an appropriate amino acid residue exposed on the surface of the VLP (i.e., an amino acid which contains a functional group capable of conjugation to the opiate conjugate).

In a particular embodiment, the coding region encodes a single-chain dimer of the coat polypeptide. In a most particular embodiment, the coding region encodes a modified single chain coat polypeptide dimer, where the modification comprises an insertion of a coding sequence at one or more amino acids at the conjugation site, which four amino acids represent a site for conjugation of the opiate conjugate as otherwise described herein. The transcription unit may contain a bacterial promoter, such as a lac promoter or it may contain a bacteriophage promoter, such as a T7 promoter.

### Synthesis

The VLPs of the present invention may be produced in vivo by introducing transcription units into bacteria, especially if transcription units contain a bacterial promoter. Alternatively, it may be synthesized in vitro in a coupled cell-free transcription/translation system.

The preferred VLP for use in the present invention is a Qbeta or Qβ VLP. These VLPs are typically made by transformation of E. coli with a plasmid expressing the Qbeta coat protein as a monomer under a lac promoter. Colonies are selected on kanamycin Luria Broth (LB) agar plates. A single colony is used to inoculate LB broth and grown overnight at 37 degrees C. This is then used to inoculate a larger culture. Cultures are shaken at 37 degrees C. for several hours until OD600 reaches 0.8. Then the expression of the Qbeta coat protein is induced with Isopropyl β-d-1-thiogalactopyranoside (IPTG) and incubated another 3 hours. Then cells are pelleted and frozen at −20 degrees C. Lysis of bacteria is then performed in isotonic buffer with sonication. VLPs are isolated by size exclusion chromatography and endotoxin is depleted with sequential Triton-X-100 phase extraction. There are many alternative methods to isolate the VLPs, which are well known in the art.

### Assembly of VLPs Encapsidating Heterologous Substances

As noted above, the VLPs of the present invention conjugate opioids on the surface of the VLPs. These VLPs may be also be assembled in combination with another substance, such as an adjuvant. Specifically, purified coat protein subunits are obtained from VLPs that have been disaggregated with a denaturant (usually acetic acid). The adjuvant is mixed with coat protein, which is then reassembled in its presence. In a particular embodiment, the substance has some affinity for the interior of the VLP and is preferably negatively charged.

In another embodiment, the adjuvant is passively diffused into the VLP through pores that naturally exist in the VLP surface. In a particular embodiment, the substance is small enough to pass through these pores and has a high affinity for the interior of the VLP.

The invention is described in additional detail in the accompanying examples presented herein below.

### EXAMPLES

Antigens can be displayed on VLPs using various methods, but one particularly effective technique the present inventors have employed is to use chemical crosslinkers to array antigens multivalently on the surface of Qβ bacteriophage VLPs. In this approach the inventors generate chemical derivatives of hydrocodone, oxycodone, and morphine such that they contain a tetraglycine linker with a terminal sulfhydryl group by incorporating a cysteinyl group. These modified opioid drugs are then be conjugated to Qβ VLPs. The vaccines are then tested by dosing and immunization schedule studies to assess the kinetics and longevity of the antibody responses elicited by the vaccine. This work investigates the ability of these vaccines to prevent opioid drug from crossing the blood-brain barrier, and prevent opioid-mediated anti-nociception in rodent models.

The goal of the present invention is the development of vaccines that protect against the effects of commonly abused opioids. Early experiments focus on identifying lead vaccines targeting each drug. Later experiments take the lead vaccines into preliminary enabling studies and expands the testing to investigate the full range of protection that these vaccines offer.

In the present invention, the inventors develop opioid vaccines by displaying drugs on a highly immunogenic Qβ bacteriophage virus-like particle (VLP)-based vaccine platform. The inventors have previously shown that a prototype VLP-based opioid vaccine can elicit high-titer serum antibodies in as few as 7 days after a single intramuscular immunization, and that mice are protected from a lethal overdose as soon as 20 days post immunization. Antibodies are long-lasting, persisting for at least 120 days after a single immunization. In further experiments, they use this same strategy to develop vaccines capable of neutralizing the effects of commonly abused opioids. The inventors develop vaccines targeting fentanyl, heroin (including its active metabolites morphine and 6-acetylmorphine), morphine, oxycodone, and hydrocodone. By the end of a first phase of development, the inventors identify lead vaccines that protect against the effects of these drugs. In further development, the inventors define the protective capacity of the lead candidates, assessing the durability of the protective response elicited by the vaccines, generation of master cell banks, upstream and downstream process development, and toxicity studies.

### Example 1

The generation of chemical derivatives of hydrocodone, oxycodone, and morphine and conjugation to the surface of Qbeta VLPs. To develop these candidate opioid vaccines, the inventors used a simple, modular vaccine design approach that takes advantage of the ability to chemically conjugate antigens at high density to the surface of Qβ bacteriophage VLPs. Oxycodone, morphine, and 6-acetylmorphine were modified to include a (Gly)₄Cys linker (FIGS. 3 and 7). Using a bifunctional crosslinker (SMPH), with sulfhydryl- and amine-reactive functional groups, the modified opioid was then chemically conjugated to the surface of purified Qβ-VLPs by virtue of the free sulfhydryl group on the Cys residue and amine-containing Lys residues that are abundantly displayed on surface of Qβ VLPs (FIG. 3). Successful conjugation was validated by SDS-PAGE analysis, showing a mobility shift in the Qβ coat protein (FIG. 3C).

Synthesis of opioid-(Gly)₄vs. Synthesis of opioid-(Gly)₄-Cys derivatives is carried out. A schematic of the synthesis is shown in attached FIG. 3. Opioid derivatives are derivatized using synthetic peptides. Peptides are synthesized manually or on a Prelude X (Gyros Protein Technologies) synthesizer. Purity (%) is determined by reverse phase HPLC, using UV detection (254 nm). Melting points are determined on a Cole-Palmer melting point apparatus. All commercial reagents and solvents are used without further purification. Characterization of the peptide and opioid derivatives are conducted using NMR spectra on an INova 500 MHz and Mercury 400 MHz nuclear magnetic resonance spectrometers. Mass spectra are recorded on a Saturn 2100 D Mass Analyzer and Bruker MALDI-TOF microflex LRF.

### Qbeta VLPs

Qbeta VLPs are made by transformation of E. coli with a plasmid expressing the Qbeta coat protein as a monomer under a lac promoter. Colonies are selected on kanamycin Luria Broth (LB) agar plates. A single colony is used to inoculate LB broth and grown overnight at 37 degrees C. This is then used to inoculate a larger culture. Cultures are shaken at 37 degrees C. for several hours until OD600 reaches 0.8. Then the expression of the Qbeta coat protein is induced with Isopropyl β-d-1-thiogalactopyranoside (IPTG) and incubated another 3 hours. Then cells are pelleted and frozen at −20 degrees C. Lysis of bacteria is then performed in isotonic buffer with sonication. VLPs are isolated by size exclusion chromatography and endotoxin is depleted with sequential Triton-X-100 phase extraction. There are many alternative methods to isolate the VLPs, which are well known in the art.

Conjugation of opioid-(Gly)₄Cys derivatives to Qbeta VLPs. Opioid-(Gly)₄-Cys derivatives are conjugated to the surface of Qbeta VLPs using a bifunctional crosslinker (e.g. SMPH) that will link the sulfhydryl group of the opiate conjugate to the surface exposed lysines of the Qbeta VLPs (FIG. 3). This strategy has been successfully for the display of diverse antigens, including peptides and full-length proteins, and others used this approach to display non-protein antigens (20). Successful conjugation of the VLPs will be confirmed by analysis of the VLPs by SDS-PAGE and Coomassie staining. FIG. 3C shows an example of a successful conjugation of a short peptide to Qbeta. The predicted molecular weight of the opioid derivatives with SMPH is ˜1.13 kDa, so successful conjugation with the Qbeta VLPs will result in Qbeta coat proteins (14.2 kDa) with a molecular weight of ˜15.3 kDa, ˜16.8 kDa, and ˜17.9 KDa corresponding to 1, 2, or 3 opioid derivatives per coat protein. These increases in molecular weight will be readily visible by SDS-PAGE and Coomassie staining. There are 180 copies of coat protein with 5 surface exposed lysines per coat protein, providing a potential for up to 900 opioid-(Gly)₄-Cys derivatives to be displayed on a single VLP.

Linker sites on the opioid molecules were chosen based on previously published successful linker attachment while also considering the potential for displaying unique structural features of the drugs to improve specificity.^{7,9,22,23} In the case of the morphine-based haptens, the base scaffold is linked through either one of the phenols (M1 and M2) or the amine (M3). See FIG. 7. In the case of the fentanyl-based haptens, the base scaffold is linked through one of the phenyl groups (F1) or off of the propanamide (F2). A fentanyl conjugate without the phenyl group will also be synthesized (F3), as was done in Raleigh et al.²³ For hydrocodone and oxycodone, the base scaffold will be linked through the phenol (C1 and O1) or the amine (C2 and O2) or the OH group (O3). In total, 14 different drug/peptide conjugates are synthesized. Linker length can be altered by changing the carbon chain or the peptide portion, but initially both the carbon chain (n=1, FIG. 3) and the peptide length (m=1, FIG. 3) is held constant initially, then varied subsequently. In the case of the morphine haptens, both free phenols and acetates will be synthesized to mimic morphine and heroin respectively. Synthesis of the morphine-based haptens is accomplished by known manipulations of the based scaffold. Synthesis of the fentanyl-based haptens will follow the standard synthesis of fentanyl analogs.²⁴ Successful drug/peptide conjugates are chemically conjugated to purified Qβ-VLPs using SMPH bifunctional or other appropriate crosslinker as described herein.

Qβ-VLPs are recombinantly expressed from a plasmid in E. coli using standard methods, purified by size-exclusion chromatography, and endotoxin depleted by sequential Triton-X100 phase extraction. Purified Qβ-VLPs are first reacted with SMPH at a 10-fold (SMPH:VLP) molar excess, followed by removal of excess SMPH by 30 kD MW cutoff centrifugation units (Amicon). Using the bifunctional crosslinker (SMPH), with sulfhydryl- and amine-reactive functional groups, the modified opioid is chemically conjugated to the surface of purified Qβ-VLPs by virtue of the free sulfhydryl group on the Cys residue and amine-containing Lys residues that are abundantly displayed on surface of QβVLPs (FIG. 3). Drug/Peptide conjugate is then added at a 10-fold (hapten:VLP) molar excess and the reaction continues overnight. The following day conjugation efficiency is evaluated by SDS-PAGE analysis. Successful conjugation is validated by SDS-PAGE analysis, showing a mobility shift in the Qβ coat protein. Drug/peptide conjugates targeting opioid haptens including morphine, 6-acetylmorphine and oxycodone (FIG. 7) are synthesized utilizing the general method set forth above and pursuant to FIG. 3 hereof. The immunogenicity of the synthesized vaccines is then assessed.

In instances where solubility of the opioid molecule poses issues for the conjugation chemistry, the oligopeptide linker may be modified to accommodate polar neutral amino acids (such as serine or threonine) in order to facilitate solubility in water. For example, oxycodone and morphine are soluble in water, while hydrocodone is soluble in ethanol and other organic solvents. When solubility becomes an issue, hydrophilic peptide linkers (i.e. SGSGC instead of GGGGC) are used to improve the solubility of hydrocodone. Hence, we begin by generating oxycodone and morphine derivatives. We have experience in modifying and optimizing the conjugation reaction and have a number of strategies that we can employ to assure successful conjugation (e.g. performing the conjugation reaction in different solvents and adjusting the ratios of the reaction components).

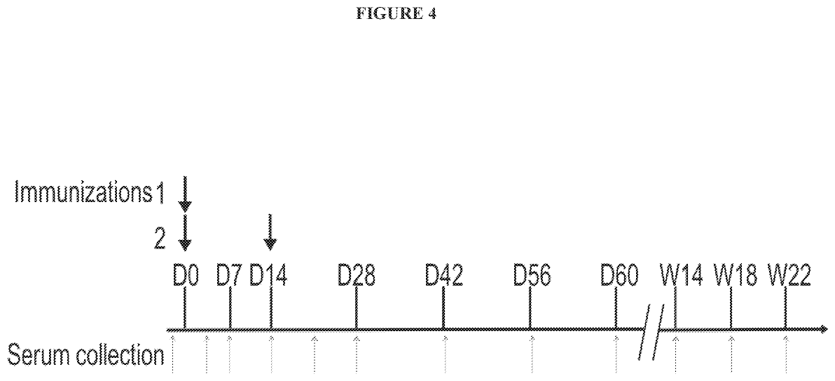

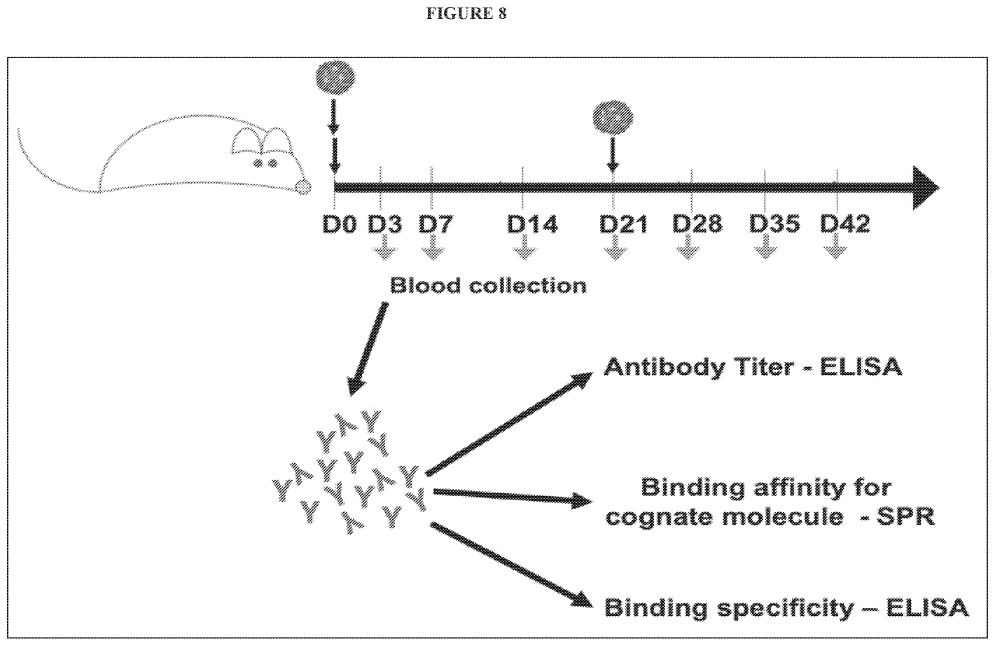

Assessing the immunogenicity of a Qbeta VLP-displayed opioid drug derivative. The vaccines engineered as described above are used to immunize mice. In general, VLP-based vaccines displaying peptide-based antigens are highly immunogenic in mice at low doses (the inventors have shown that doses as low as 500 ng can elicit high titer antibody responses)(21), however the approach to using non-protein antigens may require higher doses or may not work effectively. To assess the effects of dose and boosting on immunogenicity, mice (n=6, both sexes) are given one or two immunizations with 5, 10, or 25 μg of VLPs. In mice that receive two immunizations, the boost will occur at day 28. A schematic of the study design is shown in FIG. 4 and FIG. 8. Serum is collected by retro-orbital bleed several days prior to the first immunization, at day 3, every week thereafter until week 4, and every month thereafter until the end of the experiment. The inventors follow immunized mice for up to 8 months. The antibody responses are continued to be monitored over the life-time of the mice in order to fully characterize the longevity of the antibody response elicited by our vaccines. All serum samples are assessed for antibody titers to the corresponding drug using an ELISA. End-point dilution IgG titers are determined and compared between the groups. A control group of mice is immunized with unmodified wild-type Qbeta VLPs.

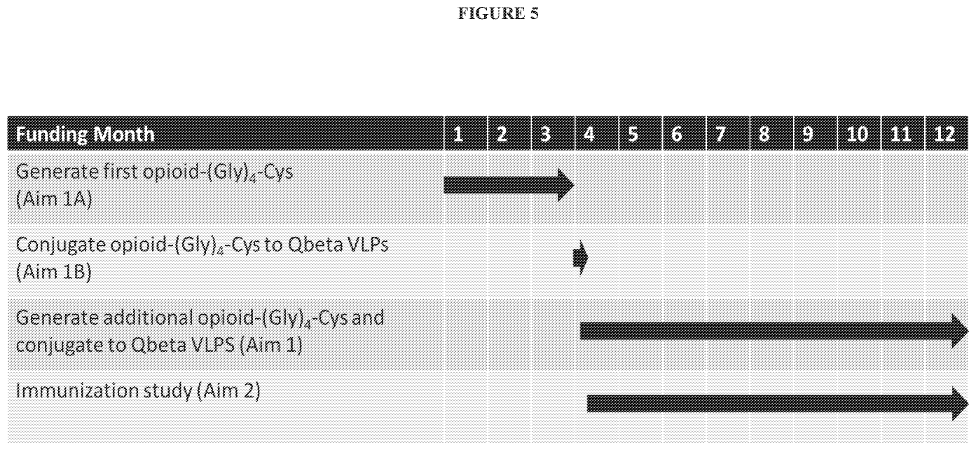

The longevity of the antibody response is assessed. The inventors collect up to 8 months of serum samples from the mice. If the vaccine is prepared ahead of the timeline of FIG. 5, the inventors immunize mice earlier in order to achieve the most extensive longevity data. If antibody titers are lower than normal for Qbeta VLP-based vaccines, the addition of adjuvants (Alum, Incomplete Freund's Adjuvant, etc.) will be used. If antibody titers drop during the course of the study, the mice are re-boosted. Boosting an immune response is an additional embodiment of the present invention.

### Example 2

### Chemical Synthesis of Vaccine Conjugates

To develop candidate opioid vaccines, the inventors used a simple, modular vaccine design approach as described in example 1, above that takes advantage of the ability to chemically conjugate antigens at high density to the surface of Qβ bacteriophage VLPs. Oxycodone, morphine, and 6-acetylmorphine were modified to include a (Gly)₄Cys linker (FIGS. 3 and 7), although numerous other opioid radicals and linkers may be used. Using the bifunctional crosslinker SMPH, with sulfhydryl- and amine-reactive functional groups, the modified opioid was chemically conjugated to the surface of purified Qβ-VLPs by virtue of the free sulfhydryl group on the Cys residue and amine-containing Lys residues that are abundantly displayed on surface of Qβ VLPs (FIG. 3). Successful conjugation for each of the opioid conjugates synthesized was validated by SDS-PAGE analysis, showing a mobility shift in the Qβ coat protein (FIG. 3).

### Assessing Immunogenicity

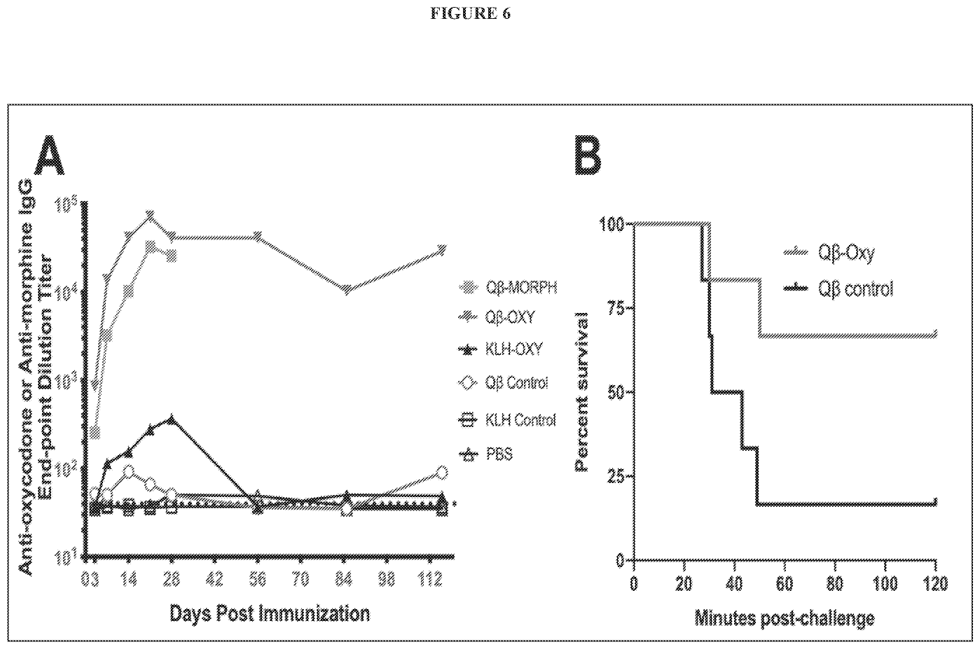

The immunogenicity of Qβ-OXY was assessed in mice and directly compared to the immunogenicity of oxycodone conjugated to KLH—a protein carrier used to produce an opioid vaccine that will soon start Phase I clinical trials.⁷ Mice were given a single 20 μg intramuscular dose of vaccine and, beginning at 3 days post-immunization, sera were collected and assessed for anti-oxycodone IgG by ELISA. Mice vaccinated with Qβ-OXY generated anti-oxycodone IgG that were detectable as soon as 3 days after immunization and peaked (at an end-point dilution titer of >10⁵) approximately 21 days post-immunization (FIG. 6A). Similar antibody kinetics are observed in mice immunized with Qβ-MORPH (FIG. 6A). Importantly, anti-oxycodone IgG antibody levels were remarkably durable. We followed these mice for four months post-immunization, and the peak titers did not diminish over that time (FIG. 6A). In contrast, mice immunized with KLH-OXY (a strategy used by competitors) generated weak IgG responses that were slower to arise and were markedly less durable (FIG. 6A).

Next, Qβ-OXY was tested to determine if the vaccine would protect mice against lethal oxycodone overdose. In a separate experiment, mice were given a single dose of Qβ-OXY (or control VLPs) and then, at 20 days post-immunization, were subcutaneously challenged with a lethal dose (426 mg/kg body weight) of oxycodone. While 84% of the control mice died within an hour of the lethal dose, two-thirds of mice immunized with Qβ-OXY survived to the end of the experiment, at 24 hours (FIG. 6B). This protection of mice from lethal overdose of an opioid after a single dose of vaccine without exogenous adjuvant provides strong preliminary data to support our opioid vaccine strategy.

### Further Experiments

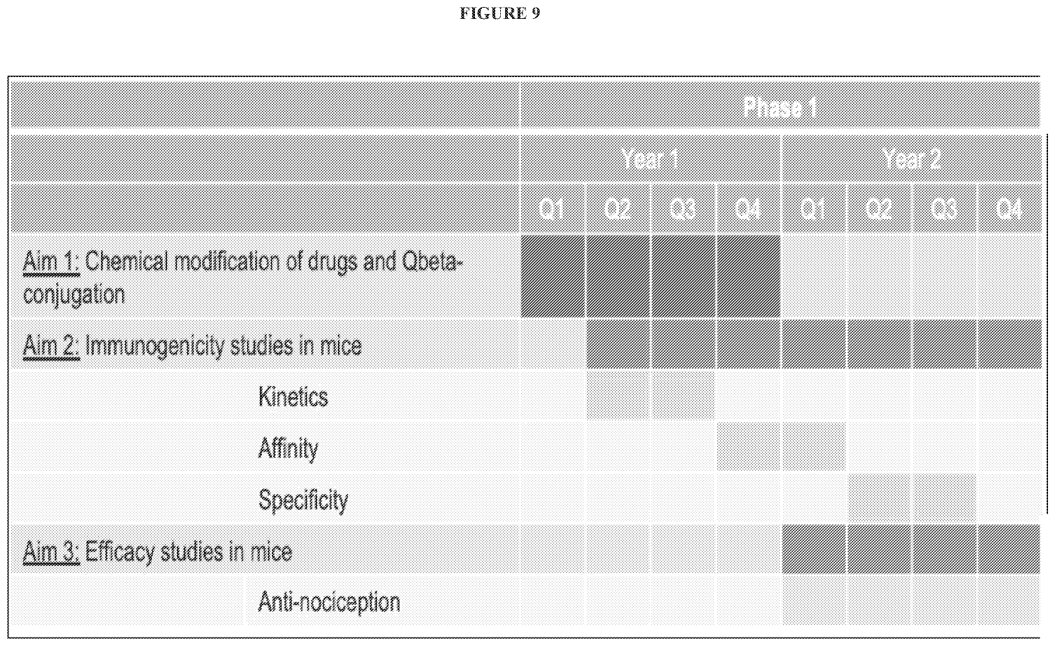

Pilot Immunization: Pilot immunization studies are performed using groups of BALB/c mice (n=12, equal numbers of males and females). Mice are immunized with 20 μg of each of the Qβ-VLP-drugs identified prepared above using the schedule shown in FIG. 9. The experiments described above demonstrated that a single intramuscular dose of vaccine without adjuvant can induce high titer antibodies by day 21 after administration (FIG. 6). However, higher titer and/or higher affinity antibodies may be achieved by giving mice an additional vaccine boost. For this reason, the effects of boosting (at D21) is also be assessed using additional groups of mice. Blood will be collected at 3, 7, 14, and 21 days, and days 28, 35, and 42 for those groups which receive two immunizations.

Antibody titer, binding affinity, and binding specificity: Specific anti-drug IgG antibodies in serum is measured by ELISA (e.g. Qβ-OXY immunized sera will be assessed for antibodies against oxycodone in ELISA). To further investigate the binding characteristics of antibodies induced by our VLP-based vaccines, peak titer serum samples are tested for serum antibodies for their binding avidity against their target drug using an SPR-based assay.²⁷ Serum from time points at which peak antibody titers are reached are also be tested for binding specificity for naloxone, methadone, buprenorphine, and endogenous opioids by modified ELISA using chaotropic agents.²⁸ FIG. 8 shows a schematic of this approach.

In general, no more than 2 immunizations are required to elicit high titer and high affinity antibodies against the target opioids. However, if necessary, further immunizations can be afforded with additional doses. Studies to investigate compatibility of VLPs with adjuvants, including ones used in human clinical trials (e.g. Advax & MF59) are ongoing and can be used if needed.

Groups Sizes and Data analysis: Group sizes for these pilot vaccination experiments were determined based on our extensive experience immunizing mice with Qβ VLP-based vaccines. VLPs generally elicit reproducible antibody titers in mice (<10-fold difference in individual titers within groups). Including 6 males and 6 females allows an analysis of the data based on the sex of the mice. All groups are compared to negative control groups immunized with unconjugated Qβ VLPs.

### Testing Vaccines for Efficacy Against Opioid-Induced Anti-Nociception.

There are multiple in vivo assays for assessing the efficacy of opioid-target vaccines in mice. Anti-nociception, respiratory depression, and lethal overdose are all endpoints of interest for opioid vaccines. Vaccine prevention of drug-induced anti-nociception is a relevant endpoint that can also be measured clinically in humans, as opposed to respiratory depression and protection against lethal overdose (which cannot be measured experimentally in humans for ethical reasons). Vaccine-elicited protection against drug-induced antinociception could be valuable for preventing relapse in patients with opioid use disorder (OUD) and is a common endpoint measured for opioid vaccines. An important outcome and interest is in establishing the anti-nociception activity of the vaccine candidates. In addition, these efficacy studies are extended to investigate overdose protection and respiratory depression.

Groups of Balb/c mice of both sexes (total N=20, as determined by power analysis, below) are immunized with each vaccine and assessed for drug-induced anti-nociception at 21 days after immunization (or at peak titer), by tail-flick assay and hot plate assay as previously described.³¹ Mice will be injected subcutaneously with drug(s), and then tested 15, 30, 50, 90, and 120 min after administration for nociception using either hot plate heated to 55° C. or tail flick using a radiant heat source. Latency to show a response (a hind paw shake or lick (for hot plate) or tail flick) will be timed by a blinded observer. Control mice will be immunized with unmodified Qβ-VLPs.

Groups Sizes and Data analysis: For efficacy studies, each group will include 10 female and 10 male mice. Based on previous studies, 10 mice/group allow for 95% power to detect a difference at p=0.05. Including male and female mice in each group will allow us to also detect a sex-based differences in response.

The timeline for these experiments is shown in FIG. 9. Because the display technology used to produce vaccines according to the present invention is modular, most vaccine constructs can be produced within 6 months.

### REFERENCES (FIRST SET)

1. Banks M L. Olson M E, Janda K D. Immunopharmacotherapies for Treating Opioid Use Disorder. Trends Pharmacol Sci. 2018; 39(11):908-11. doi: 10.1016/j.tips.2018.08.001. PubMed PMID: 30343725.
2. Kimishima A. Wenthur C J, Zhou B, Janda K D. An Advance in Prescription Opioid Vaccines: Overdose Mortality Reduction and Extraordinary Alteration of Drug Half-Life. ACS Chem Biol. 2017; 12(1):36-40. Epub 2016 Nov. 29. doi: 10.1021/acschembio.6b00977. PubMed PMID: 28103678.
3. Pravetoni M. Le Naour M, Harmon T M, Tucker A M, Portoghese P S, Pentel P R. An oxycodone conjugate vaccine elicits drug-specific antibodies that reduce oxycodone distribution to brain and hot-plate analgesia. J Pharmacol Exp Ther. 2012; 341(1):225-32. Epub 2012 Jan. 18. doi: 10.1124/jpet.111.189506. PubMed PMID: 22262924; PMCID: PMC3310692.
4. Pravetoni M, Vervacke J S, Distefano M D, Tucker A M, Laudenbach M, Pentel P R. Effect of currently approved carriers and adjuvants on the pre-clinical efficacy of a conjugate vaccine against oxycodone in mice and rats. PLoS One. 2014; 9(5):e96547. Epub 2014 May 5. doi: 10.1371/journal.pone.0096547. PubMed PMID: 24797666; PMCID: PMC4010527.
5. Raleigh M D, Laudenbach M, Baruffaldi F, Peterson S J, Roslawski M J, Birnbaum A K, Carroll F I, Runyon S P. Winston S, Pentel P R, Pravetoni M. Opioid Dose- and Route-Dependent Efficacy of Oxycodone and Heroin Vaccines in Rats. J Pharmacol Exp Ther. 2018; 365(2):346-53. Epub 2018 Mar. 13. doi: 10.1124/jpet. 117.247049. PubMed PMID: 29535156; PMCID: PMC5884377.
6. Raleigh M D, Baruffaldi F, Peterson S J, Le Naour M, Harmon T M, Vigliaturo J R, Pentel P R, Pravetoni M. Vaccination reduces fentanyl distribution to the brain and fentanyl-induced toxicity in mice and rats: a potential role for a prophylactic vaccine against fentanyl-induced overdose. J Pharmacol Exp Ther. 2018. Epub 2018 Nov. 8. doi: 10.1124/jpet.118.253674. PubMed PMID: 30409833.
7. Caldeira J, Bustos J, Peabody J, Chackerian B, Peabody D S. Epitope-Specific Anti-hCG Vaccines on a Virus Like Particle Platform. PLoS One. 2015; 10(10):e0141407. doi: 10.1371/journal.pone.0141407. PubMed PMID: 26516771 PMCID: PMC4627648.
8. Chackerian B. Virus-like particles: flexible platforms for vaccine development. Expert Rev Vaccines. 2007; 6(3):381-90. Epub 2007 Jun. 5. doi: 10.1586/14760584.6.3.381. PubMed PMID: 17542753.
9. Chackerian B, Durfee M R, Schiller J T. Virus-like display of a neo-self antigen reverses B cell anergy in a B cell receptor transgenic mouse model. J Immunol. 2008; 180(9):5816-25. Epub 2008 Apr. 22. doi: 180/9/5816 [pii]. PubMed PMID: 18424700.
10. Chackerian B. Virus-like particle based vaccines for Alzheimer disease. Hum Vaccin. 2010; 6(11):926-30. Epub 2010 Sep. 25. doi: 12655 [pii] 10.4161/hv.7.1.12655. PubMed PMID: 20864801.
11. Crossey E. Amar M J A, Sampson M, Peabody J, Schiller J T. Chackerian B, Remaley A T. A cholesterol-lowering VLP vaccine that targets PCSK9. Vaccine. 2015; 33(43):5747-55. Epub 2015 Sep. 26. doi: 10.1016/j.vaccine.2015.09.044. PubMed PMID: 26413878; PMCID: PMC4609631.
12. Frietze K M, Peabody D S, Chackerian B. Engineering virus-like particles as vaccine platforms. Curr Opin Virol. 2016; 18:44-9. doi: 10.1016/j.coviro.2016.03.001. PubMed PMID: 27039982.
13. O'Rourke J P, Daly S M, Triplett K D, Peabody D, Chackerian B. Hall P R. Development of a mimotope vaccine targeting the Staphylococcus aureus quorum sensing pathway. PLoS One. 2014; 9(11):e111198. Epub 2014 Nov. 8. doi: 10.1371/journal.pone.0111198. PubMed PMID: 25379726; PMCID: 4224382.
14. Peabody D S, Manifold-Wheeler B, Medford A, Jordan S K, do Carmo Caldeira J. Chackerian B. Immunogenic display of diverse peptides on virus-like particles of RNA phage MS2. J Mol Biol. 2008; 380(1):252-63. doi: 10.1016/j.jmb.2008.04.049. PubMed PMID: 18508079; PMCID: PMC2481506.
15. Tumban E, Peabody J, Peabody D S, Chackerian B. A universal virus-like particle-based vaccine for human papillomavirus: longevity of protection and role of endogenous and exogenous adjuvants. Vaccine. 2013; 31(41):4647-54. doi: 10.1016/j.vaccine.2013.07.052. PubMed PMID: 23933337; PMCID: PMC3785330.
16. Mohsen M O, Zha L, Cabral-Miranda G, Bachmann M F. Major findings and recent advances in virus-like particle (VLP)-based vaccines. Semin Immunol. 2017; 34:123-32. Epub 2017 Sep. 5.doi: 10.1016/j.smim.2017.08.014. PubMed PMID: 28887001.
17. Farlow M R, Andreasen N, Riviere M E, Vostiar I, Vitaliti A, Sovago J, Caputo A, Winblad B. Graf A. Long-term treatment with active Aβ immunotherapy with CAD106 in mild Alzheimer's disease. Alzheimers Res Ther. 2015; 7(1):23. Epub 2015 Apr. 27. doi: 10.1186/s13195-015-0108-3. PubMed PMID: 25918556; PMCID: PMC4410460.
18. Kündig T M, Senti G. Schnetzler G, Wolf C, Prinz Vavricka B M. Fulurija A, Hennecke F, Sladko K, Jennings G T, Bachmann M F. Der p 1 peptide on virus-like particles is safe and highly immunogenic in healthy adults. J Allergy Clin Immunol. 2006; 117(6):1470-6. Epub 2006 Apr. 27. doi: 10.1016/j.jaci.2006.01.040. PubMed PMID: 16751015.
19. Tissot A C, Maurer P, Nussberger J. Sabat R, Pfister T, Ignatenko S, Volk H D, Stocker H, Muller P, Jennings G T, Wagner F. Bachmann M F. Effect of immunisation against angiotensin II with CYT006-AngQb on ambulatory blood pressure: a double-blind, randomised, placebo-controlled phase IIa study. Lancet. 2008; 371(9615):821-7. Epub 2008 Mar. 11. doi: 10.1016/s0140-6736(08)60381-5. PubMed PMID: 18328929.
20. Maurer P, Jennings G T, Willers J. Rohner F. Lindman Y, Roubicek K, Renner W A, Müller P, Bachmann M F. A therapeutic vaccine for nicotine dependence: preclinical efficacy, and Phase I safety and immunogenicity. Eur J Immunol. 2005; 35(7):2031-40. doi: 10.1002/eji.200526285. PubMed PMID: 15971275.
21. Tumban E, Peabody J, Tyler M, Peabody D S, Chackerian B. VLPs displaying a single L2 epitope induce broadly cross-neutralizing antibodies against human papillomavirus. PLoS One. 2012; 7(11):e49751. Epub 2012 Nov. 19. doi: 10.1371/journal.pone 0049751. PubMed PMID: 23185426; PMCID: PMC3501453.

### REFERENCES (SECOND SET)

1. Pravetoni M, Comer S D. Development of vaccines to treat opioid use disorders and reduce incidence of overdose. Neuropharmacology. 2019 Nov. 1; 158:107662. PMCID: PMC6919211
2. Banks M L, Olson M E, Janda K D. Immunopharmacotherapies for Treating Opioid Use Disorder. Trends Pharmacol Sci. 2018; 39(11):908-911. PMCID: PMC6472257
3. Wheeler E, Jones T S, Gilbert M K, Davidson P J, Centers for Disease Control and Prevention (CDC). Opioid Overdose Prevention Programs Providing Naloxone to Laypersons—United States, 2014. MMWR Morb Mortal Wkly Rep. 2015 Jun. 19; 64(23):631-635. PMCID: PMC4584734
4. Bonese K F, Wainer B H, Fitch F W, Rothberg R M. Schuster C R. Changes in heroin self-administration by a rhesus monkey after morphine immunisation. Nature. 1974 Dec. 20; 252(5485):708-710. PMID: 4474602
5. Volkow N D, Collins F S. The Role of Science in Addressing the Opioid Crisis. N Engl J Med. 2017 Jul. 27; 377(4):391-394. PMID: 28564549
6. Xu A, Kosten T R. Current status of immunotherapies for addiction. Ann N Y Acad Sci. 2020 Mar. 8; PMID: 32147860
7. Baruffaldi F, Raleigh M D, King S J, Roslawski M J, Birnbaum A K, Hassler C, Carroll F I, Runyon S P, Winston S, Pentel P R, Pravetoni M. Formulation and Characterization of Conjugate Vaccines to Reduce Opioid Use Disorders Suitable for Pharmaceutical Manufacturing and Clinical Evaluation. Mol Pharm. 2019 Jun. 3; 16(6):2364-2375. PMCID: PMC6598681
8. Sulima A, Jalah R, Antoline J F G, Torres O B, Imler G H, Deschamps J R. Beck Z, Alving C R, Jacobson A E, Rice K C, Matyas G R. A Stable Heroin Analogue That Can Serve as a Vaccine Hapten to Induce Antibodies That Block the Effects of Heroin and Its Metabolites in Rodents and That Cross-React Immunologically with Related Drugs of Abuse. J Med Chem. 2018 Jan. 11; 61(1):329-343. PMCID: PMC5767880
9. Bremer P T, Kimishima A, Schlosburg J E, Zhou B, Collins K C, Janda K D. Combatting Synthetic Designer Opioids: A Conjugate Vaccine Ablates Lethal Doses of Fentanyl Class Drugs. Angew Chem Int Ed Engl. 2016 Mar. 7; 55(11):3772-3775. PMCID: PMC4860013
10. Mohsen M O, Zha L, Cabral-Miranda G, Bachmann M F. Major findings and recent advances in virus-like particle (VLP)-based vaccines. Semin Immunol. 2017; 34:123-132. PMID: 28887001
11. Chackerian B, Lowy D R, Schiller J T. Conjugation of a self-antigen to papillomavirus-like particles allows for efficient induction of protective autoantibodies. J Clin Invest. 2001 August; 108(3):415-423. PMCID: PMC209354
12. Chackerian B. Lowy D R, Schiller J T. Induction of autoantibodies to mouse CCR5 with recombinant papillomavirus particles. Proc Natl Acad Sci USA. 1999 Mar. 2; 96(5):2373-2378. PMCID: PMC26791
13. Chackerian B. Durfee M R, Schiller J T. Virus-like display of a neo-self antigen reverses B cell anergy in a B cell receptor transgenic mouse model. J Immunol. 2008 May 1; 180(9):5816-5825. PMCID: PMC3493123
14. Tumban E, Peabody J, Peabody D S, Chackerian B. A pan-HPV vaccine based on bacteriophage PP7 VLPs displaying broadly cross-neutralizing epitopes from the HPV minor capsid protein. L2. PLoS One. 2011; 6(8):e23310. PMCID: PMC3157372
15. Frietze K M, Peabody D S, Chackerian B. Engineering virus-like particles as vaccine platforms. Curr Opin Virol. 2016; 18:44-49. PMCID: PMC4983494
16. Huang X, Wang X. Zhang J, Xia N, Zhao Q. Escherichia coli-derived virus-like particles in vaccine development. NPJ Vaccines. 2017; 2:3. PMCID: PMC5627247
17. Maphis N M, Peabody J, Crossey E, Jiang S, Jamaleddin Ahmad F A, Alvarez M, Mansoor S K, Yaney A. Yang Y, Sillerud L O, Wilson C M, Selwyn R, Brigman J L, Cannon J L, Peabody D S, Chackerian B, Bhaskar K. Qβ Virus-like particle-based vaccine induces robust immunity and protects against tauopathy. NPJ Vaccines. 2019; 4:26. PMCID: PMC6547647
18. Daly S M, Joyner J A, Triplett K D, Elmore B O, Pokhrel S, Frietze K M, Peabody D S, Chackerian B, Hall P R. VLP-based vaccine induces immune control of Staphylococcus aureus virulence regulation. Sci Rep. 2017 Apr. 4; 7(1):637. PMCID: PMC5429642
19. Crossey E, Amar M J A, Sampson M, Peabody J, Schiller J T, Chackerian B, Remaley A T. A cholesterol-lowering VLP vaccine that targets PCSK9. Vaccine. 2015 Sep. 26 ed. 2015 October; 33(43):5747-5755.
20. Tumban E, Muttil P. Escobar C A A, Peabody J, Wafula D, Peabody D S, Chackerian B. Preclinical refinements of a broadly protective VLP-based HPV vaccine targeting the minor capsid protein, L2. Vaccine. 2015 Jun. 26; 33(29):3346-3353. PMCID: PMC4468037
21. O'Rourke J P, Daly S M. Triplett K D, Peabody D. Chackerian B. Hall P R. Development of a mimotope vaccine targeting the Staphylococcus aureus quorum sensing pathway. PLoS One. 2014; 9(11):e111198. PMCID: PMC4224382
22. Anton B, Salazar A. Flores A, Matus M, Marin R, Hernandez J-A, Leff P. Vaccines against morphine/heroin and its use as effective medication for preventing relapse to opiate addictive behaviors. Hum Vaccin. 2009 April; 5(4):214-229. PMID: 19242094
23. Raleigh M D, Baruffaldi F, Peterson S J, Le Naour M, Harmon T M, Vigliaturo J R, Pentel P R, Pravetoni M. A Fentanyl Vaccine Alters Fentanyl Distribution and Protects against Fentanyl-Induced Effects in Mice and Rats. J Pharmacol Exp Ther. 2019; 368(2):282-291. PMCID: PMC6346379
24. Valdez C A, Leif R N. Mayer B P. An efficient, optimized synthesis of fentanyl and related analogs. PLoS One. 2014; 9(9):e108250. PMCID: PMC4169472
25. Stowe G N, Schlosburg J E, Vendruscolo L F. Edwards S, Misra K K, Schulteis G, Zakhari J S, Koob G F, Janda K D. Developing a vaccine against multiple psychoactive targets: a case study of heroin. CNS Neurol Disord Drug Targets. 2011 December; 10(8):865-875. PMCID: PMC3327724
26. Maynard J A, Maassen C B M, Leppla S H, Brasky K, Patterson J L, Iverson B L, Georgiou G. Protection against anthrax toxin by recombinant antibody fragments correlates with antigen affinity. Nat Biotechnol. 2002 June; 20(6):597-601. PMID: 12042864
27. Lynch H E, Stewart S M, Kepler T B, Sempowski G D, Alam S M. Surface plasmon resonance measurements of plasma antibody avidity during primary and secondary responses to anthrax protective antigen. J Immunol Methods. 2014 February; 404:1-12. PMCID: PMC4104170
28. Staats H F, Fielhauer J R, Thompson A L, Tripp A A, Sobel A E, Maddaloni M, Abraham S N, Pascual D W. Mucosal targeting of a BoNT/A subunit vaccine adjuvanted with a mast cell activator enhances induction of BoNT/A neutralizing antibodies in rabbits. PLoS One. 2011 Jan. 27; 6(1):e16532. PMCID: PMC3029387
29. Jalah R, Torres O B, Mayorov A V, Li F, Antoline J F G, Jacobson A E. Rice K C, Deschamps J R, Beck Z, Alving C R. Matyas G R. Efficacy, but not antibody titer or affinity, of a heroin hapten conjugate vaccine correlates with increasing hapten densities on tetanus toxoid, but not on CRM197 carriers. Bioconjug Chem. 2015 Jun. 17; 26(6):1041-1053. PMCID: PMC4472500
30. Pedersen G K, Höschler K, Øie Solbak S M, Bredholt G. Pathirana R D, Afsar A. Breakwell L, Nostbakken J K, Raae A J, Brokstad K A, Sjursen H, Zambon M, Cox R J. Serum IgG titres, but not avidity, correlates with neutralizing antibody response after H5N1 vaccination. Vaccine. 2014 Jul. 31; 32(35):4550-4557. PMID: 24950357
31. Miller L K, Hou X, Rodriguiz R M, Gagnidze K, Sweedler J V, Wetsel W C, Devi L A. Mice deficient in endothelin-converting enzyme-2 exhibit abnormal responses to morphine and altered peptide levels in the spinal cord. J Neurochem. 2011 December; 119(5):1074-1085. PMCID: PMC3217172
32. Chackerian B. Peabody D S. Factors That Govern the Induction of Long-Lived Antibody Responses. Viruses. 2020 Jan. 7; 12(1). PMCID: PMC7019776
33. Safacian M, Porras C, Pan Y, Kreimer A, Schiller J T, Gonzalez P. Lowy D R, Wacholder S, Schiffman M, Rodriguez A C, Herrero R, Kemp T. Shelton G, Quint W, van Doom L-J, Hildesheim A. Pinto L A, CVT Group. Durable antibody responses following one dose of the bivalent human papillomavirus L1 virus-like particle vaccine in the Costa Rica Vaccine Trial. Cancer Prev Res (Phila). 2013 November; 6(11):1242-1250. PMCID: PMC7605443
34. Boyer E W. Management of opioid analgesic overdose. N Engl J Med. 2012 Jul. 12; 367(2):146-155. PMCID: PMC3739053
35. Wilson N. Kariisa M, Seth P, Smith H, Davis N L. Drug and Opioid-Involved Overdose Deaths—United States, 2017-2018. MMWR Morb Mortal Wkly Rep. 2020 Mar. 20; 69(11):290-297. PMID: 32191688
36. Campen M J, Shimoda L A, O'Donnell C P. Acute and chronic cardiovascular effects of intermittent hypoxia in C57BL/6J mice. J Appl Physiol (1985). 2005 November; 99(5):2028-2035. PMID: 16002771
37. Campen M J, Tagaito Y, Jenkins T P, Smith P L, Schwartz A R, O'Donnell C P. Phenotypic differences in the hemodynamic response during REM sleep in six strains of inbred mice. Physiol Genomics. 2002 Dec. 3; 11(3):227-234. PMID: 12388788
38. Seagrave J, Campen M J, McDonald J D, Mauderly J L, Rohr A C. Oxidative stress, inflammation, and pulmonary function assessment in rats exposed to laboratory-generated pollutant mixtures. J Toxicol Environ Health A. 2008; 71(20):1352-1362. PMID: 18704828
39. Tuet W Y, Pierce S A, Racine M C, Tressler J, McCranor B J, Sciuto A M, Wong B. Changes in murine respiratory dynamics induced by aerosolized carfentanil inhalation: Efficacy of naloxone and naltrexone. Toxicol Lett. 2019 November; 316:127-135. PMID: 31539569

1. A composition comprising: (a) a virus-like particle (VLP) comprising a bacteriophage coat protein; and (b) at least one conjugated opiate determinant; wherein said opioid determinant is displayed on said virus-like particle, and wherein said determinant comprises a conjugated opiate derived from an opioid compound.

2. The composition of claim 1, wherein said opiate conjugate determinant is displayed at one or more lysine residues at the A-B loop, N-terminus or carboxy terminus of said bacteriophage coat protein.

3. The composition of claim 1 or 2, wherein said opiate conjugate determinant is displayed at high density on the surface of said VLP.

4. The composition of any of claims 1-3 wherein the bacteriophage coat protein is a coat protein derived from Qbeta or AP205 bacteriophage.

5. The composition of claim 1, 3 or 4 wherein said bacteriophage coat protein is a coat protein derived from Qbeta bacteriophage.

6. The composition of claim 1, 3 or 43 wherein said bacteriophage coat protein is a coat protein derived from AP205 bacteriophage.

7. The composition according to any one of claims 1-6 wherein said opiate conjugate determinant is displayed at one or more lysine residues on the surface of the bacteriophage.

8. The composition according to claim 2 or 7 wherein said opiate conjugate is displayed on said bacteriophage at said lysine residues by covalently binding an opioid molecule to said lysine residues through a linker group.

9. The composition according to claim 8 wherein said linker group comprises an oligopeptide covalently bonded to a crosslinker.

10. The composition according to claim 9 wherein said oligopeptide is covalently bonded to an electrophilic or nucleophilic group on the opioid molecule and the crosslinker is bonded to said lysine residues on the surface of said bacteriophage.

11. The composition according to claims 8-10 wherein said oligopeptide is a 4 to 15 mer oligopeptide comprising neutral amino acid residues bonded to an amine group on said opioid molecule.

12. The composition according to claim 11 wherein said neutral amino acid residues are selected from the group consisting of glycine, alanine, valine, leucine, isoleucine, phenylalanine, tryptophan, methione, proline, serine and mixtures thereof.

13. The composition according to claim 11 or 12 wherein said neutral amino acids are selected from the group consisting of glycine, serine and mixture thereof.

14. The composition according to any of claims 11-13 wherein said amino acid residues are glycine residues.

15. The composition according to any of claims 10-14 wherein said electrophilic group is a carbonyl group or a vinyl group of said opioid molecule.

16. The composition according to any of claims 10-14 wherein said nucleophilic group is a hydroxyl group or amine group of said opioid molecule.

17. The composition of any of claims 1-16 wherein said opiate conjugate comprises an opiate radical selected from the group consisting of a radical of codeine, fentanyl, hydrocodone, hydromorphone, meperidine, methadone, morphine, oxycodone, diacetylmorphine (heroin), hydroxylmorphine, 2,4-dinitrophenylmorphine, 6-methyldihydromorphine, 6-methylenedihydrodesoxymorphine, 6-acetyldihydromorphine, chloranaltrexamine, chloroxymorphamine, dexomorphine, dihydromorphine, ethyldihydromorphine, hydromorphinol, methyldesorphine, morphine methyl bromide, n-phenylnordesomorphine, N-phenylnormorphine, 6-nicotinoyldihydromorphine, acetylpropionylmorphine, 3,6-dibutanoylmorphine, dibutyrylmorphine, dibenzoylmorphine, diformylmorphine, diacetyl morphine (herone), dipropanooymorphine, nicomorphine, 6-monoacetylecodeine, benzylmorphine, codeine methylbromide, desocodeine, dimethylmorphine, ethyldihydromorphine, heterocodeine, dihydrocodeine, isocodeine, morpholinylethylmorphine, myrophine, transisocodeine, acetylcodone, acetylmorphone, dihydrocodeine, hydroxycodeine, codeinone, hydrocodone, hydromorphone, morphinol, morphinone and mixtures thereof.

18. The composition of any of claims 1-17 wherein said opiate conjugate comprises an opiate radical selected from the group consisting of a radical of codeine, fentanyl, hydrocodone, hydromorphone, meperidine, methadone, morphine, oxycodone and diacetylmorphine (heroin).

19. The composition according to any of claims 1-17 wherein said opiate conjugate comprises an opiate radical selected from the group consisting of fentanyl, heroin, morphine, 6-acetylmorphine, oxycodone, and hydrocodone.

20. The composition according to any of claims 1-17 wherein said opiate conjugate comprises an opiate radical selected from the group consisting of fentanyl, heroin, morphine or oxycodone.

21. A population of virus-like particles according to any of claims 1-20.

22. A pharmaceutical composition comprising a population of virus-like particles according to claim 21 in combination with a pharmaceutically acceptable carrier, additive and/or excipient.

23. The composition according to claim 22 which is formulated as a vaccine for administration to a subject or patient.

24. The composition according to claim 23 wherein said vaccine comprises an adjuvant.

25. A method for enhancing an immune response against an opiate compound in a patient or subject in need comprising introducing the composition of claim 22 or 23 into said subject or patient, wherein an enhanced immune response against said opiate compound is produced in said patient or subject.

26. The method of claim 25, wherein the composition is prophylactic for an opiate induced disorder.

27. A method of inducing an immunogenic response in a patient or subject comprising administering a composition according to claim 22 or 23 to said patient or subject.

28. A method for treating or inhibiting opioid use disorder or a symptom thereof in a patient or subject in need comprising administering to said patient a composition according to claim 22 or 23 to said patient or subject.

29. The method of claim 28 wherein said disorder is opiate dependency.

30. The method of claim 28 wherein said symptom is nausea, vomiting, weakened immune system, slow breathing rate/respiratory depression, coma, increased risk of infectious disease including hepatitis, hallucinations, collapsed veins or clogged blood vessels, risk of choking, increased tolerance to opioids, an inability to stop or reduce usage, narcolepsy, extreme weight loss or gain, anxiety, sweating, insomnia, agitation, tremors, muscle aches, nausea, vomiting, diarrhea or extreme mental and physical discomfort.

31. A method for treating or reducing the likelihood of an opioid overdose or a symptom thereof in a patient or subject in need comprising administering to said patient a composition according to claim 22 or 23 to said patient or subject.

32. The method according to claim 31 wherein said symptom is nausea, vomiting, slow breathing rate, coma, hallucinations, collapsed veins or clogged blood vessels, risk of choking, narcolepsy, extreme weight loss or gain, anxiety, sweating, insomnia, agitation, tremors, muscle aches, nausea, vomiting, diarrhea or extreme mental and physical discomfort.

33. A method for treating or reducing the likelihood of drug-induced antinociception in a patient or subject in need comprising administering to said patient or subject a composition according to claim 22 or 23 to said patient or subject.

34. The method according to claim 33 which treats drug-induced antinociception.

35. The method according to claim 33 which reduces the likelihood of drug-induced antinociception.
